(19) United States
(12) Patent Application Publication
(10) Pub. No.: US 2005/0102161 A1
(43) Pub. Date: May 12, 2005

(54) Secure network gateway for accessible patient data and transplant donor data

(76) Inventors: Robert Michael Kalthoff, Cincinnati, OH (US); Rudolph Siegel, Cincinnati, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 10/813,918

(22) Filed: Mar. 31, 2004

Related U.S. Application Data
(60) Provisional application No. 60/459,205, filed on Mar. 31, 2003.

(51) Int. Cl. A61K 31/34; G06F 17/60

(52) U.S. Cl. 705/2

(57) ABSTRACT

A method of accessing transplant donor data from a remote location, the method comprising the steps of: (a) accessing a database over a network containing transplant donor data that includes information specific to a potential transplant donor; (b) reviewing at least one of information specific to the potential transplant donor, cause of death of the potential transplant donor, and time of death of the potential transplant donor; and (c) acting on the reviewed transplant donor data to establish qualification to at least one of an organ and a tissue available for transplant.

### CROSS REFERENCE TO RELATED APPLICATIONS

The present application claims the benefit of U.S. Provisional Patent Application Ser. No. 60/459,205, filed Mar. 31, 2003.

### BACKGROUND

The present invention is related to information systems and more specifically to organ and/or tissue transplant information systems utilized by hospitals, organ and/or tissue procurement organizations, transplant centers, and the like. The present invention reduces the time to disseminate organ and tissue transplant information and allows multiple parties to view the same organ and/or tissue transplant data concurrently, without compromising the secure nature of the gateway through which the data is accessed.

In 1954 a kidney was removed from one identical twin and placed in the other setting the stage for a revolutionary advancement in medicine that would launch an entire industry and would save countless lives. Today, not only can kidneys be transplanted, but also hearts, livers, pancreata, lungs and intestines, as well as tissue. Over 24,000 human organs alone are transplanted each year.

With the increase in transplants has come an increase in the number of patients waiting for transplants. Over 80,000 people in the U.S. are waiting for organ transplants. Sadly, 17 people on this waiting list die each day due to a lack of organs.

Initially, nurses within organ transplant hospitals took on the task of finding suitable donors to donate the needed organs. As the number of organ transplant hospitals grew, the organ procurement function moved outside of these hospitals to resolve conflict of interest issues and for efficiency reasons. Eventually, the organ procurement function required more than a single nurse and so the Organ Procurement Organization was formed. It was also realized that tissue procurement and organ procurement could be combined into the same organization, known as an Organ Procurement Organization, however, some tissue procurement may be performed by separate tissue procurement organizations.

Today, there are 59 Organ Procurement Organizations or OPOs serving more than 261 transplant hospitals in the United States. All OPOs are non-profit and 90% are not directly affiliated with any hospital.

When a potential donor is identified at one of nearly 4,000 hospitals in the United States, which may include a patient on life support with no brain activity, a referral call is placed to the OPO that serves that donor hospital. An initial assessment is done over the phone to determine if the patient has the potential to be an organ donor, and potentially a tissue donor. If the OPO determines that there is a potential for that patient to be a donor, an OPO Procurement Transplant Coordinator or PTC will be dispatched to that donor hospital.

Once on site, the PTC will attempt to obtain family member consent for that patient to donate their organs and/or tissue. Once consent is granted, the PTC will begin to run a series of tests to determine the medical suitability for donation. Depending on the test results, the PTC will attempt to get verbal acceptance of the placement of one or more organs (several organs such as the liver can be split and transplanted into multiple recipients) from that donor.

Today, nearly all the 59 OPOs enter the medical information pertaining to a donor onto a 16 plus-page manual paper-based form (See FIGS. 4-17). Hardcopy documents, such as an EKG form or Consent form are included with the donor medical data form which together makes up the donor medical chart. Once some of the medical information is completed on the form, an organ recipient list is requested via a telephone call to OPTN—the Organ Procurement Transplant Network for each organ that the PTC is attempting to place. The Coordinator will not attempt to place organs based upon medical insuitability or for other reasons. OPTN is the U.S. Government's designated organization that maintains the national organ recipient list. OPTN is not currently a part of the tissue recovery, tissue processing, tissue placement and tissue transplant process. Human tissue is usually recovered from the donor, frozen, stored then shipped, then processed into usable form then placed and shipped to requesting tissue transplant hospitals, then transplanted to awaiting tissue recipient patients.

OPTN faxes the organ recipient list that contains transplant center or referring OPO contact information to that PTC at the donor hospital. Then, the PTC will begin calling listed transplant hospitals (or their representing OPO) to begin the placement process. The PTC reads off the medical information for that donor—a process that may take over 20 minutes per call. The first organ transplant center on the list is offered the organ, has (usually) 1 hour to indicate if they want that organ or not. If the PTC is not contacted by the offered transplant center within approximately 1 hour from the time the offer was made, the PTC will skip that transplant center and it's recipients and the PTC will then call the next transplant center on the list. This entire organ placement process can take 12-20 hours-meanwhile, the donor's health can quickly deteriorate at which point the donor will need to be rushed into an operating room to retrieve those organs that have been placed. If the heart, lungs and/or liver have not yet been placed, there is little chance that those organs will get placed. It is effectively a race against time where lives hang in the balance. Nationally, only 3 of the (typical) 7 organs per donor are actually transplanted. The other 4 organs are discarded, used for research or buried with the donor.

Since the 1950's, the medical advances in human organ transplantation have increased at an amazing rate. Liver transplants, for example, 15 years ago took 15 hours. Today, 3-hour liver transplant operations are not uncommon. Anti-rejection drugs, used to reduce the possibility of transplanted organ rejection, have also improved the quality of life of the organ recipient in the last 30 years.

Since the advent of the first Organ Procurement Organization, there has been no improvement in the capturing and dissemination of the medical data of the donor and medical data pertaining directly to the donor's organs. Today, at nearly all of the OPO's and donor hospitals in the U.S., donor medical data is still captured with a pen and paper and is disseminated via telephone calls or by fax from the PTC to the offered transplant centers. During a typical donor case, the PTC will spend 6 to 12 hours calling organ transplant centers and reading dozens of pieces of medical information pertaining to the donor over and over again. Because organ transplantation is a race against time, not all of the donor information may be conveyed to the transplant centers due to lack of time.

Further, transplant surgeons have performed numerous “dry runs” (when the transplant team travels to the donor hospital to retrieve the organ, only to come away empty-handed) because the doctors at the donor hospitals who read the X-Rays, bronchoscopies and/or echocardiograms or other medical images are often times not as skilled in interpreting the health of that organ. Each dry run costs a transplant center upwards of $10,000. Therefore, there is a need to provide transplant surgeons and transplant centers with organ donor data and medical images accessible by more than one party at a time to reduce the frequency of dry runs and reduce costs at the transplant centers and to improve the organ offer/acceptance process.

### SUMMARY OF THE INVENTION

The present invention is related to information systems and network systems and, more specifically, to organ and/or tissue transplant information systems and organ and/or tissue network systems utilized by hospitals, organ and/or tissue procurement organizations, transplant centers, and the like. The present invention reduces the time to disseminate organ and tissue transplant information and allows multiple parties to view the same transplant data concurrently, without compromising the secure nature of the gateway through which the data is accessed. In one exemplary embodiment of the present invention, an Organ Transplant Response System (OTRS) automates the organ and tissue placement process and streamlines the notification, verification, and matching of potential transplantable organs and tissue with potential transplant recipients, thereby saving countless lives by enabling more organs and tissue to be placed from the (already consented) organ donors and by increasing the quantity, quality, and speed of donor data and/or donor images communicated to intended third parties.

The system in a more detailed exemplary embodiment includes a client application and a server application operative to pass files from a client computer to a server computer via dial-up modem/landline, Ethernet connection or through a wireless card or computer-phone or smart phone capable of handling data transmission. As wireless transmission speeds and wireless device capability expands, so too will the capability of the present invention.

OTRS, in addition to accommodating electronic donor information, may also accommodate electronic transplant recipient information and include software to identify and display potentially matching and potentially exclusionary items in order to assist in the match of donor and recipient data. Such a dual data source allows the transplant surgeon to review medical data comparing their patient and the donor all in the same or in different computer systems. Thus, an increased number of organs and/or tissue are accepted, with increased speed of acceptance and with a heightened post-operative condition for the transplant recipient. The entire process utilizing the OTRS may be paperless or include one or more hardcopies that may be amendable to digital scanning or other electronic data storage technique.

The OTRS system may also include a qualitative and quantitative tracking aspect to enable long term monitoring of donor recipients and the placement and success rates of transplant centers. The OTRS system may further include a searchable database feature to enable an intended third party to search donor records and review those records most relevant to a particular set of circumstances.

It is a first aspect of the present invention to provide a method of accessing transplant donor data from a remote location, the method comprising the steps of: (a) accessing a database over a network containing transplant donor data that includes at least one of: (i) information specific to a potential transplant donor, (ii) cause of death of a potential transplant donor, and (iii) time of death of a potential transplant donor; (b) reviewing at least one of information specific to the potential transplant donor, cause of death of the potential transplant donor, and time of death of the potential transplant donor; and (c) acting on the reviewed transplant donor data to establish qualification to at least one of an organ and a tissue available for transplant.

It is a second aspect of the present invention to provide a method of gathering and inputting transplant donor data to a database, the method comprising the steps of: (a) compiling a transplant donor record specific to a transplant donor; (b) accessing a remote database capable of storing a plurality of transplant donor records; and (c) uploading or downloading the transplant donor record to the remote database.

It is a third aspect of the present invention to provide a method of gathering and displaying transplant donor data, the method comprising the steps of: (a) compiling a transplant donor record specific to a transplant donor; (b) transmitting an electronic version of the transplant donor record; and (c) displaying the transplant donor record using the electronic version of the transplant donor record.

It is a fourth aspect of the present invention to provide a method of organizing and making available transplant donor data, the method comprising the steps of: (a) providing a secure database to store transplant donor data; (b) providing access to a selective third party to at least one of upload and view transplant donor data and download and view transplant donor data; and (c) generating an authorization code required to access the transplant donor data by the selective third party.

It is a fifth aspect of the present invention to provide a method of gathering and inputting transplant donor data to a database in the form of a pure data system, the method comprising the steps of: (a) providing a series of data input options into which transplant donor data may be input to create an transplant donor record, the series of data input options including at least one of edit transplant donor data, update transplant donor data, delete transplant donor data, and submit transplant donor data; (b) inputting transplant donor data into at least one of the series of data input options to create the transplant donor record; (c) transmitting the transplant donor record to a transplant donor database; and (d) availing the transplant donor record via the Internet.

It is a sixth aspect of the present invention to provide a method of gathering transplant donor data, the method comprising the step of utilizing a computer operatively coupled to a scanner, where the computer has at least one electronic transplant donor form adapted to be manipulatable to input transplant donor data using at least one of keystrokes, digital handwriting, and scanned images, where the computer includes software to facilitate the uploading of the transplant donor data to a remote database over a network connection, the remote database including a remote digital processing device, such that the remote database is accessible by an intended third party.

It is a seventh aspect of the present invention to provide a transplant information system to facilitate the dissemination of information pertaining to transplantable organs and tissue from a donor hospital to a transplant center, the system comprising: (a) a database accessible by a procurement organization representative and a transplant center representative, the database including transplant donor data; and (b) a secure network through which the transplant donor data is accessed by at least one of the procurement organization representative and the transplant center representative.

### BRIEF DESCRIPTION OF THE DRAWINGS

FIG. 1 is a schematic diagram of a first exemplary embodiment of the present invention;

FIG. 2 is a schematic diagram of an exemplary architecture of the first exemplary embodiment of the present invention;

FIG. 3 is a schematic diagram of an exemplary data communication process of the first exemplary embodiment of the present invention;

FIGS. 4-17 are exemplary hardcopy donor forms that may be utilized to comprise a donor record in accordance with the present invention;

FIGS. 18-46 are exemplary electronic donor forms that may be utilized to comprise a donor record in accordance with the present invention;

FIG. 47 is an exemplary screen shot of an exemplary transplant coordinator login in accordance with the present invention;

FIG. 48 is an exemplary screen shot of an exemplary transplant coordinator welcome screen in accordance with the present invention;

FIG. 49 is an exemplary screen shot of an exemplary transplant coordinator new donor record entry screen in accordance with the present invention;

FIG. 50 is an exemplary screen shot of an exemplary third party logon in accordance with the present invention;

FIG. 51 is an exemplary schematic diagram of an exemplary contact web in accordance with the present invention;

FIG. 52 is an exemplary schematic diagram of an exemplary security process for data associated with the present invention;

FIG. 53 is an exemplary screen shot of an exemplary OTRS coordinator's OPO subscriber listing in accordance with the present invention;

FIG. 54 is an exemplary screen shot of an exemplary OPO administrator logon in accordance with the present invention;

FIG. 55 is an exemplary screen shot of an exemplary OPO administrator's view of donor records in accordance with the present invention;

FIG. 56 is an exemplary screen shot of an exemplary OPO administrator's list of coordinators in accordance with the present invention;

FIG. 57 is an exemplary screen shot of an exemplary OPO administrator's list of exemplary questions regarding donor data in accordance with the present invention;

FIG. 58 is an exemplary screen shot of an exemplary OPO administrator's list of exemplary questions that may be edited in accordance with the present invention;

FIG. 59 is an exemplary screen shot of an exemplary OPO administrator's edit screen to amend data regarding donor hospitals in accordance with the present invention;

FIG. 60 is an exemplary screen shot of an exemplary OPO administrator's list of donor hospitals in accordance with the present invention;

FIG. 61 is an exemplary screen shot of an exemplary OPO administrator's edit screen to amend the individuals receiving notification in accordance with the present invention;

FIG. 62 is an exemplary screen shot of an exemplary OPO administrator's list of individuals receiving notification in accordance with the present invention; and

FIG. 63 is an exemplary schematic diagram of an alternate exemplary embodiment of the present invention.

### DETAILED DESCRIPTION

The exemplary embodiments of the present invention are described and illustrated below to encompass systems and methods for inputting, organizing, and making available organ and/or tissue donor data to interested third parties. Of course, it will be apparent to those of ordinary skill in the art that the preferred embodiments discussed below are exemplary in nature and may be reconfigured without departing from the scope and spirit of the present invention. However, for clarity and precision, the exemplary embodiments may include one or more optional features that one of ordinary skill may recognize as not being a requisite to fall within the scope of the present invention. In addition, it is to be understood that organ data includes organ donor data and tissue data include tissue donor data. Still further, it is to be understood that while one or more of the exemplary embodiments discussed below may not be explained concurrently with respect to organs and tissue, it is within the scope of the invention to provide such concurrent systems and methods. Likewise, an exemplary embodiment including a concurrent system and method for organs and tissue does not necessitate such a dual functionality. For instance, the OTRS system may include tissue data to the exclusion of organ data.

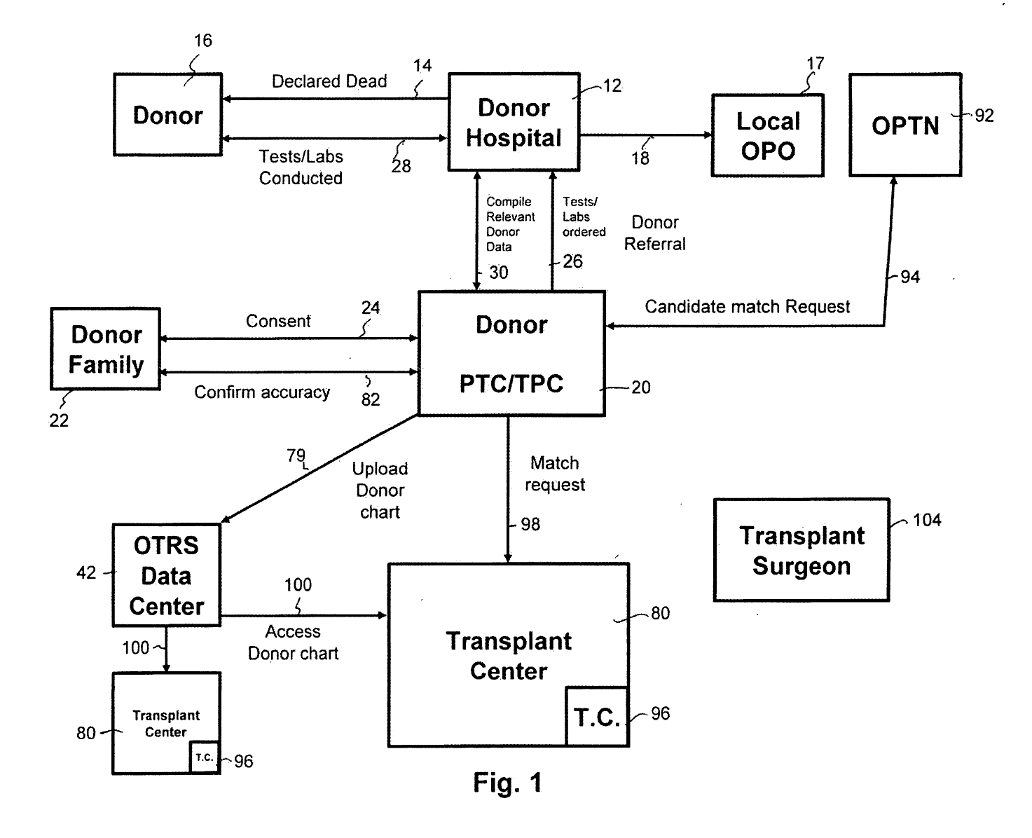

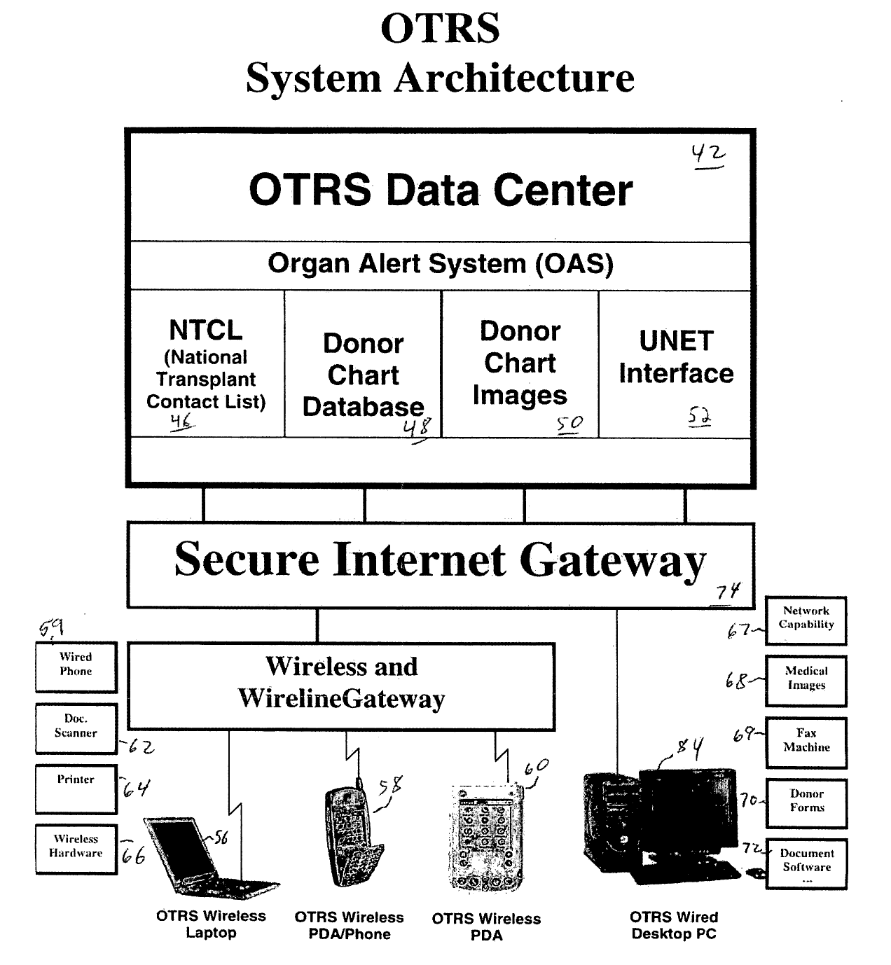

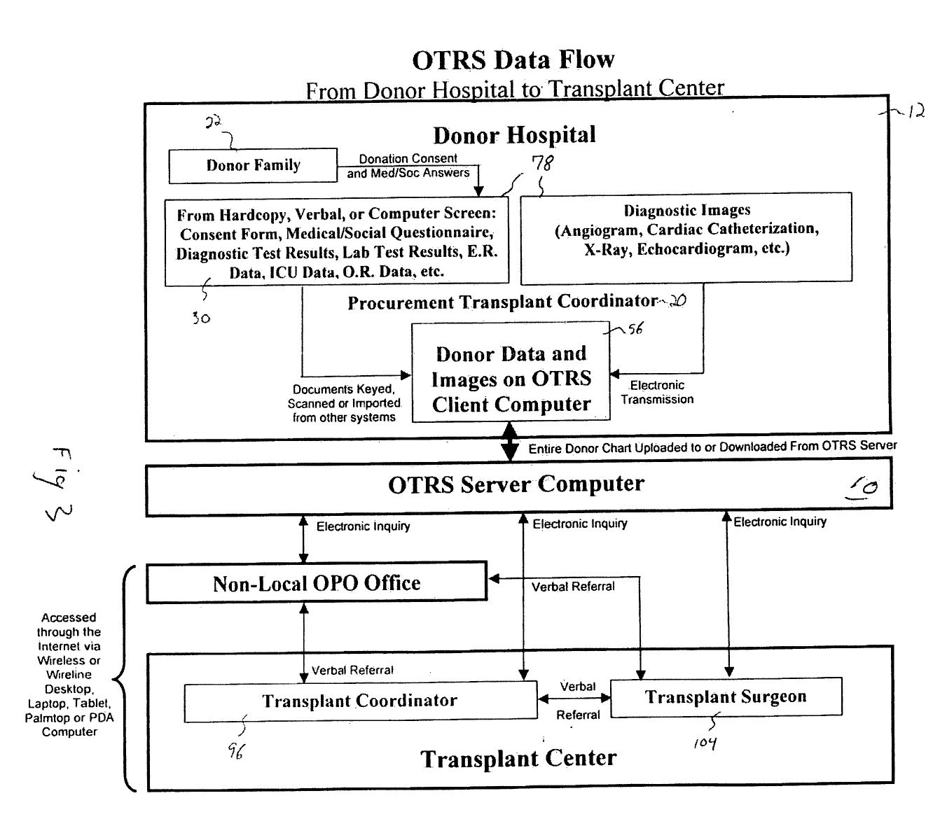

Referencing FIGS. 1-3, an exemplary sequence of events may occur between the time a patient arrives at a hospital or other medical care facility and the time that one or more organs from the consented donor are transplanted into recipient patients utilizing an Organ Transplant Response System (OTRS) 10 in accordance with the present invention (See FIG. 2). It is important to note that the exemplary sequence of events need not occur in the exact order discussed herein, nor include each and every event in order to fall within the scope of the invention.

An exemplary starting point includes admitting a patient to a hospital or other medical care facility 12 for treatment of a medical problem. Treatment may include any form of medical attention from providing medical care to better the condition of the injured patient to pronouncing the patient dead. Types of injuries that are common to organ and tissue donors include, without limitation, gunshot wounds, trauma from car wrecks, and brain bleeds. Obviously, even if the injured patient is near death, the hospital staff will do everything to treat the injuries and place the patient on a path of continued healing. However, some injuries cannot be overcome by the medical knowledge and/or treatments currently utilized, thereby resulting in the death of such patients.

The determination of death 14 may be rather sudden in cases of mass trauma or may be extended in cases where the patient's condition deteriorates for some number of days prior to being declared dead. In an exemplary circumstance, the patient/donor 16 will be assumed to be involved in a motor vehicle accident (MVA) with injuries leading to a comatose state. Typically, one or two separate doctors generally conduct neurological examinations of the injured patient every 6-12 hours. If one or several neurological examinations confirm no brain activity and machines are providing the patient with oxygen and nutrients in order to keep the vital organs functioning, the doctors may call their representing Organ Procurement Organization (OPO) 17; i.e., a donor referral 18. On occasion, the OPO 17 is notified prior to a brain death declaration if the donor hospital 12 determines that a brain death declaration is imminent.

An OPO call center, generally open around the clock, receives the donor referral via telephone call or other type of communication 18 and forwards it to a Donation PTC or Procurement Transplant Coordinator (PTC) 20. In some cases, a Tissue Procurement Coordinator (TPC) will be called to perform the Coordination duties needed to document the medical suitability of recovering tissue from the donor 16. In some cases the PTC and TPC are the same person. The PTC and/or TPC 20, which may be a nurse, will call the hospital 12 from which the donor referral 18 originated and ask a hospital medical professional a series of questions to determine if the decedent 16 is a potential organ and/or tissue donor. Organs utilized for donation purposes include, without limitation, hearts, lungs, livers, intestines, kidneys, and pancreata. Tissue utilized for donation purposes includes, without limitation, heart valves, corneas, bone, ligaments, tendons, skin, and muscle. If the answers to the questions asked by the PTC and/or TPC 20 result in the determination that the decedent 16 is a potential organ and/or tissue donor, the PTC and/or TPC 20 will arrive at the hospital 12 where the potential donor 16 is located. Typically, potential donors 16, if located in the emergency room, are transferred to the intensive care unit (ICU) in anticipation of the arrival of the PTC and/or TPC 20.

In particular states, for example Ohio, injured patients 16 are presumed to consent to organ and tissue removal for transplant, commonly referred to as “presumed consent” states. In such circumstances, the PTC and/or TPC 20 may tell family members 22 of the decedent 16 that the doctors and hospital personnel are fulfilling the wishes of their loved one by using the decedent's organs and/or tissue to provide a better life for another human being and no signed consent form is required. Conversely, if a hospital 12 is not located within the jurisdiction of a presumed consent state, the PTC and/or TPC 20 is required to obtain a signature from a donor family member 22 on a consent form to effectuate the organ and/or tissue consent 24. At the point at which the consent 24 is acknowledged and the patient 16 has been determined to be clinically brain dead, the financial burden of caring for the decedent 16 is usually taken on by the OPO and/or tissue recovery agency 17 at that time.

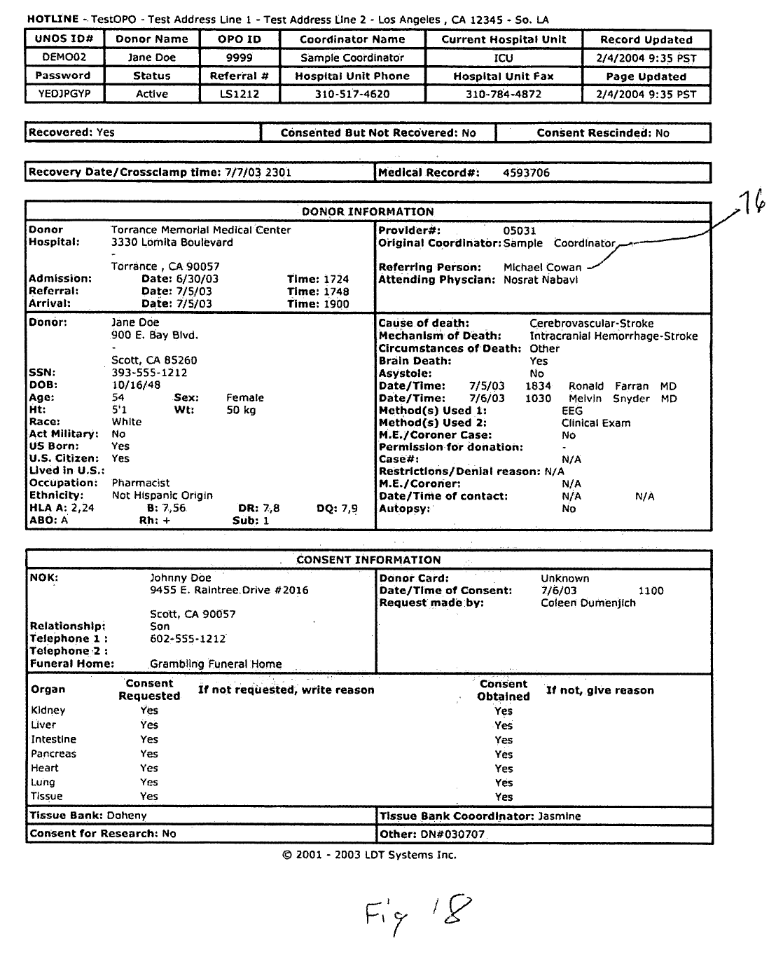

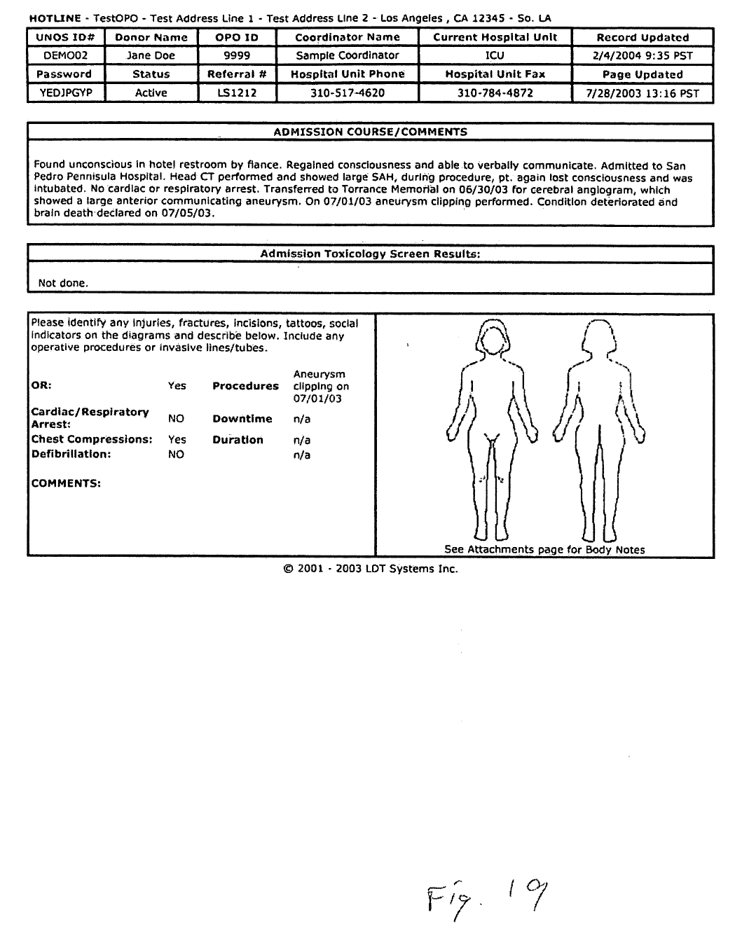

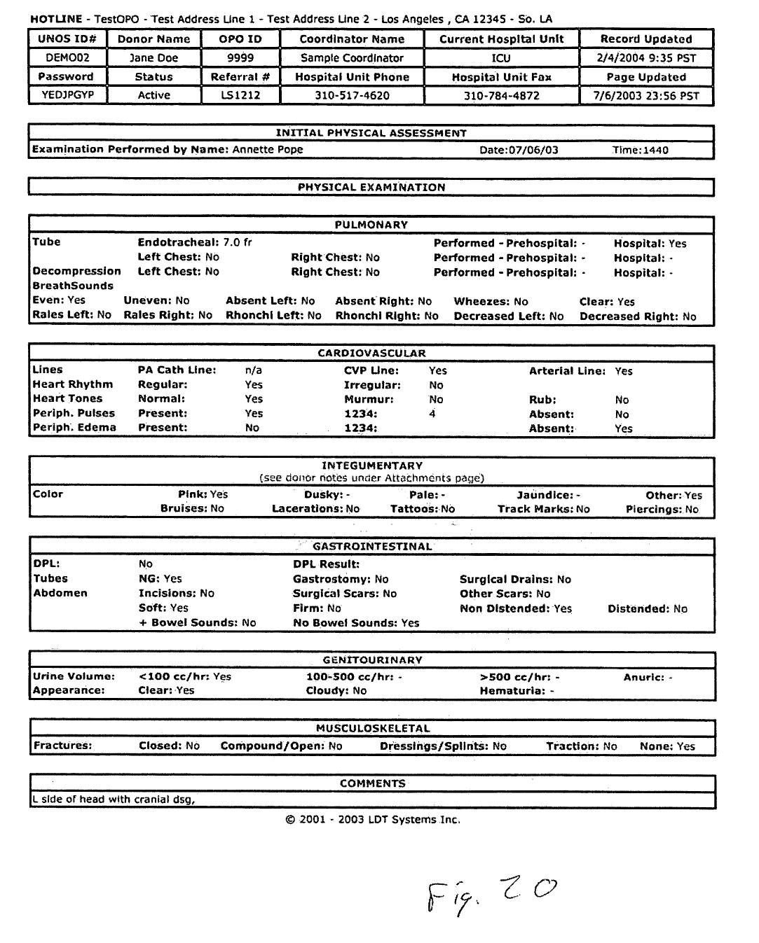

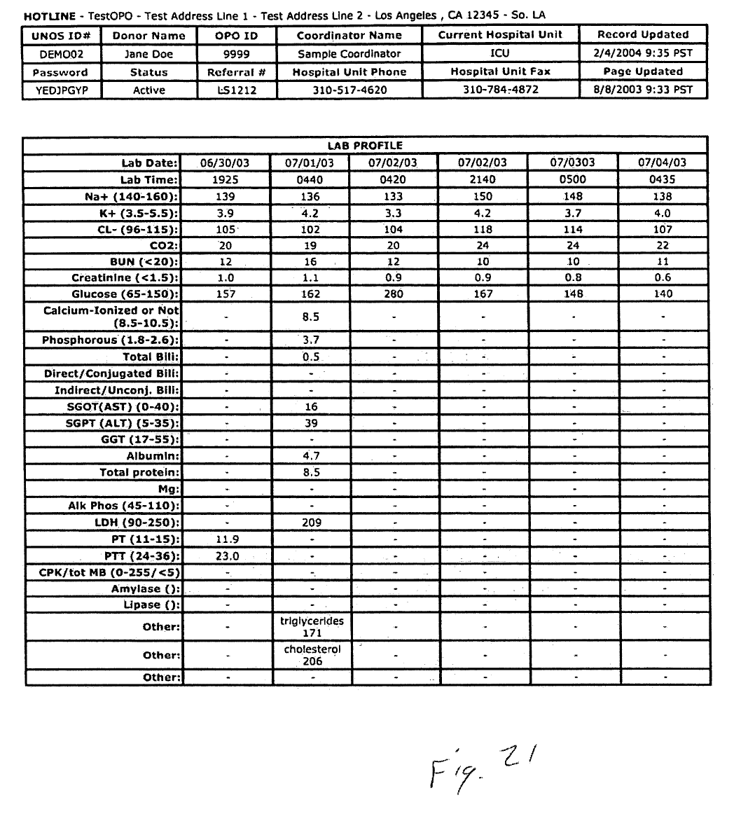

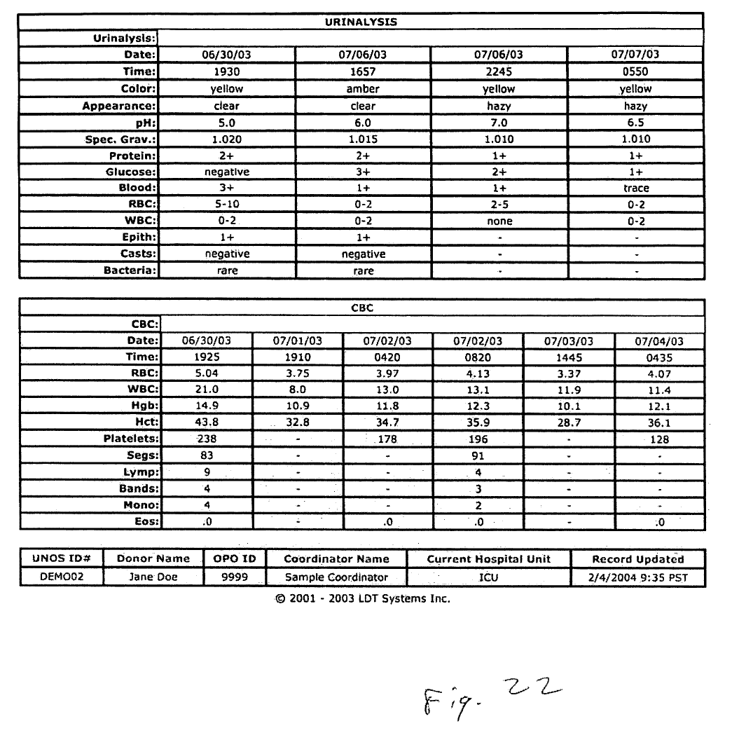

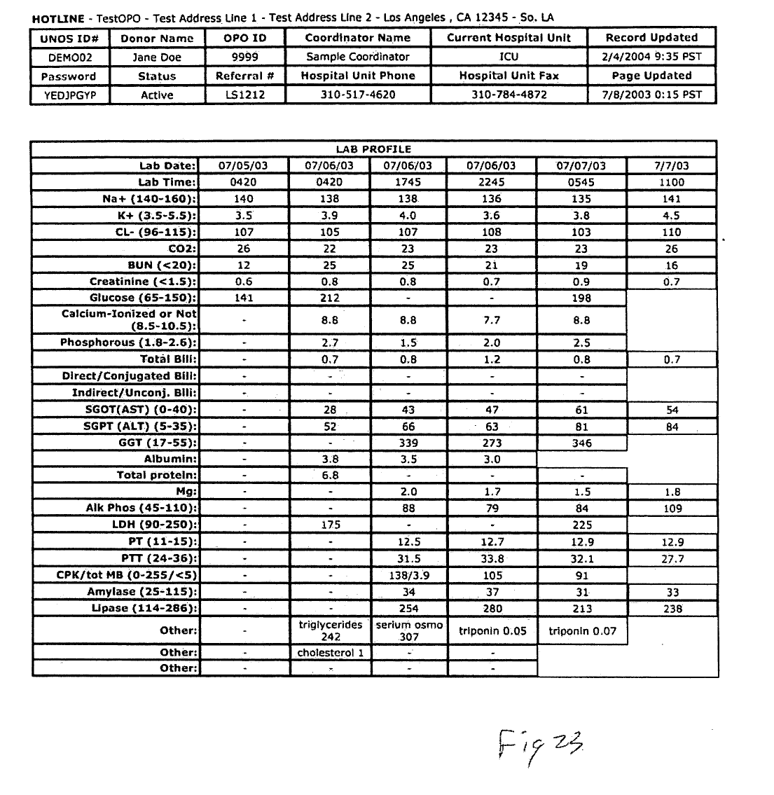

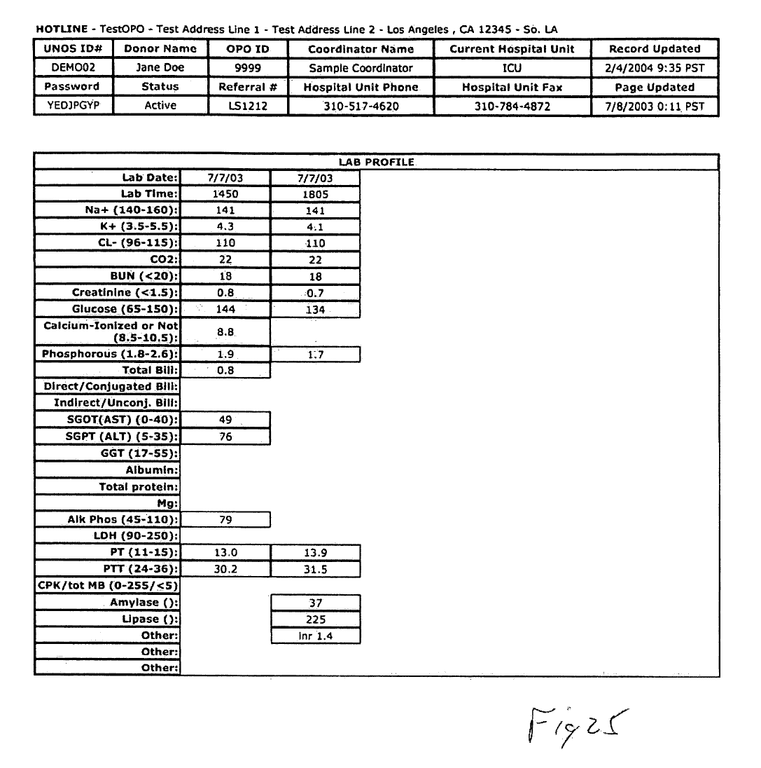

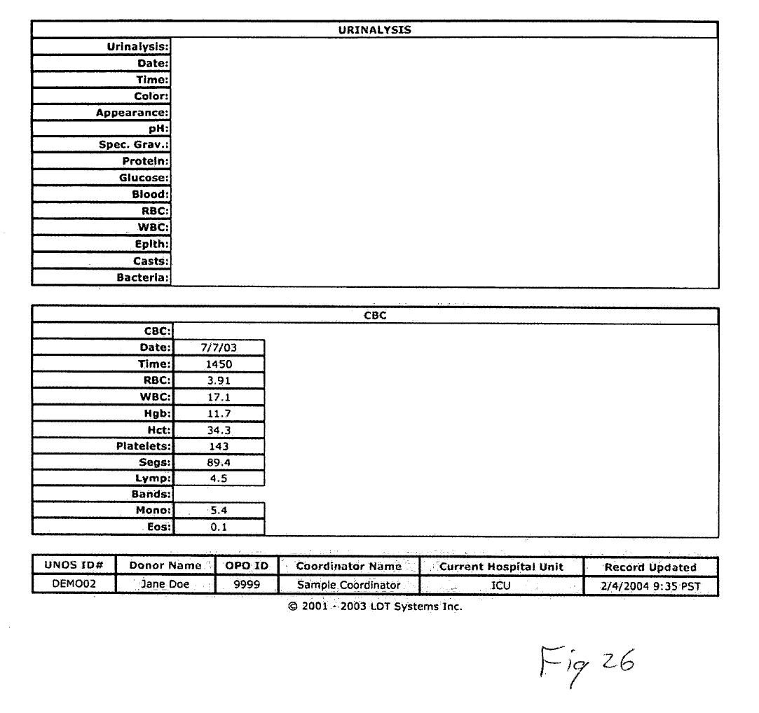

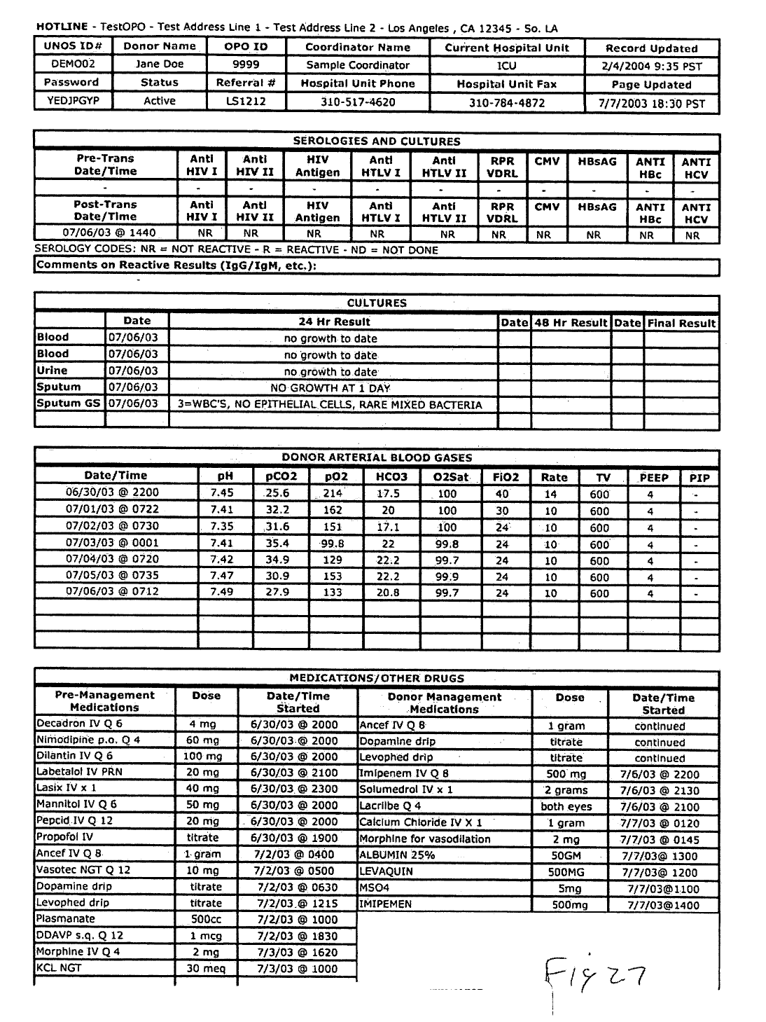

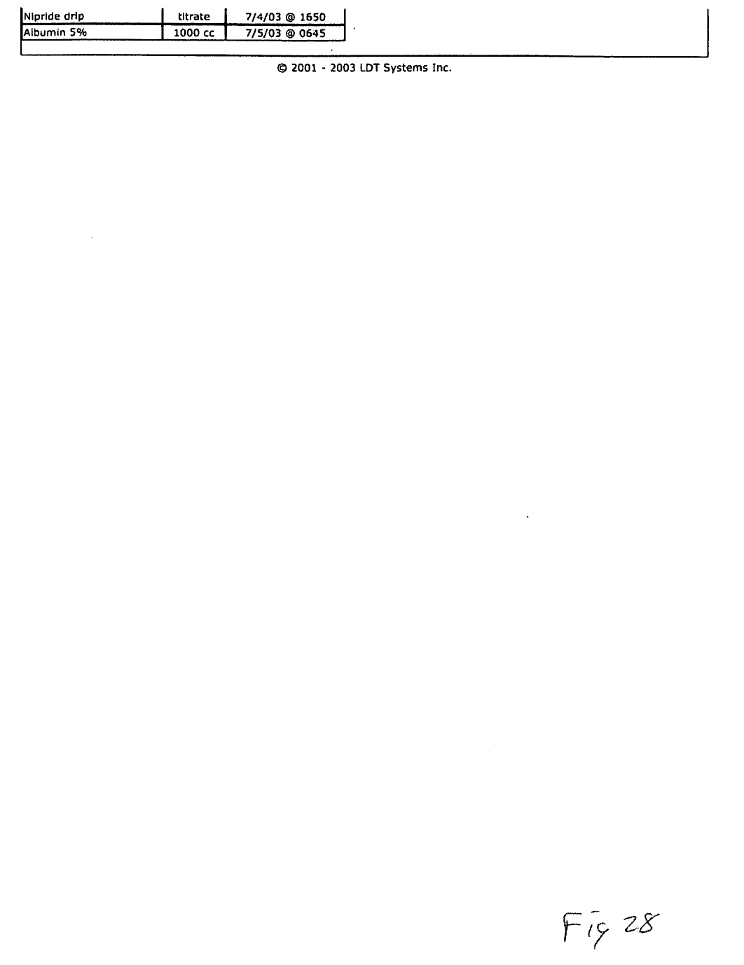

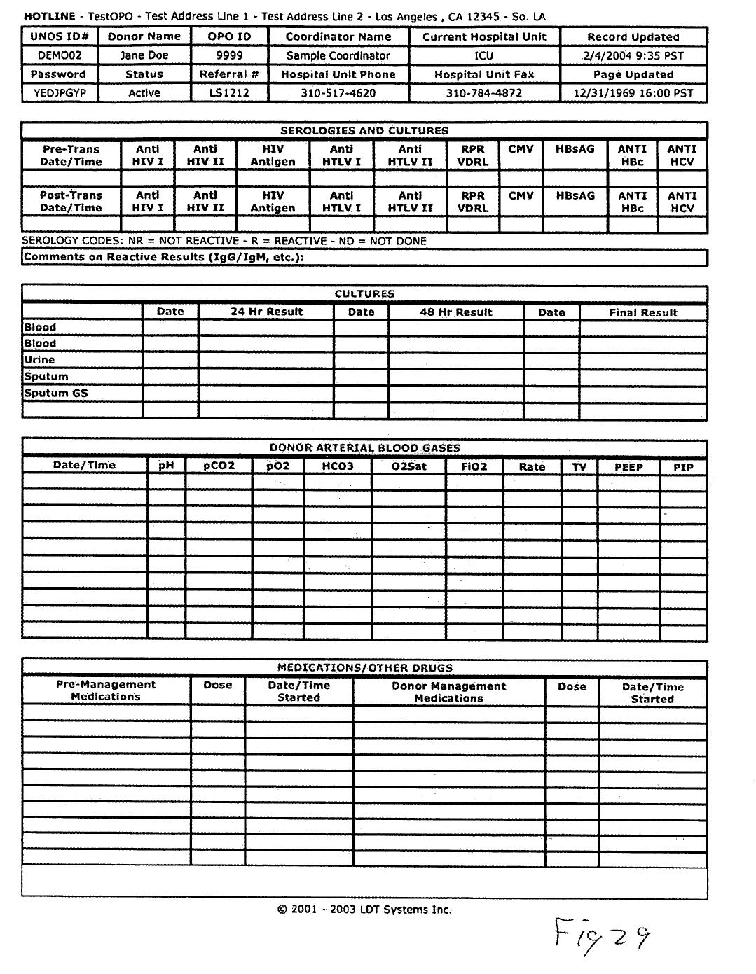

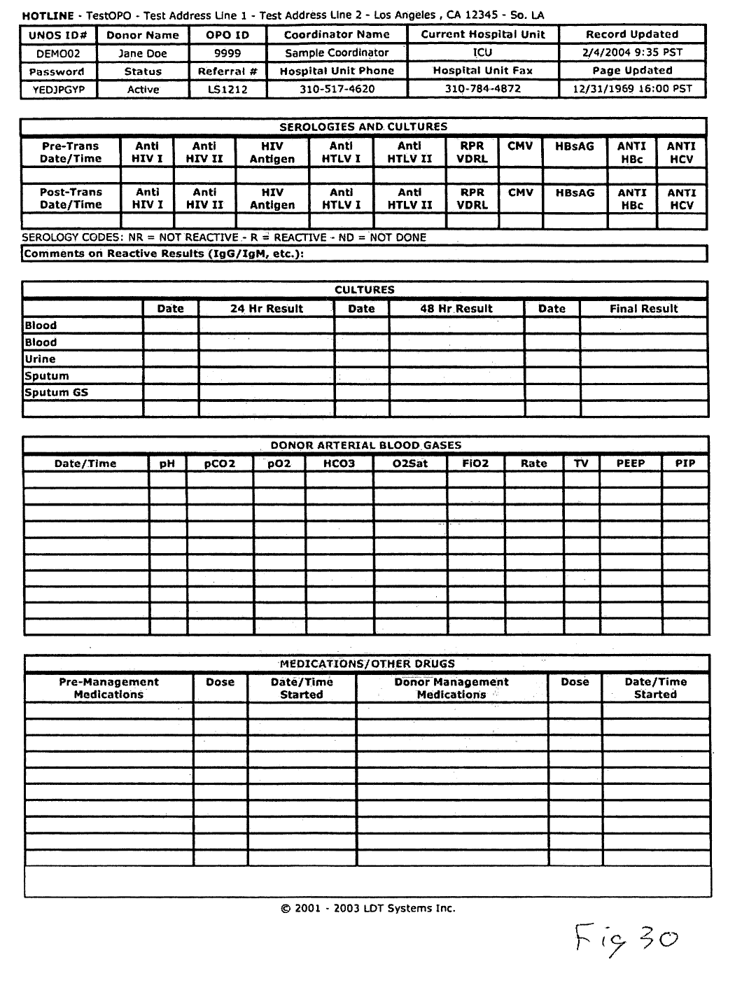

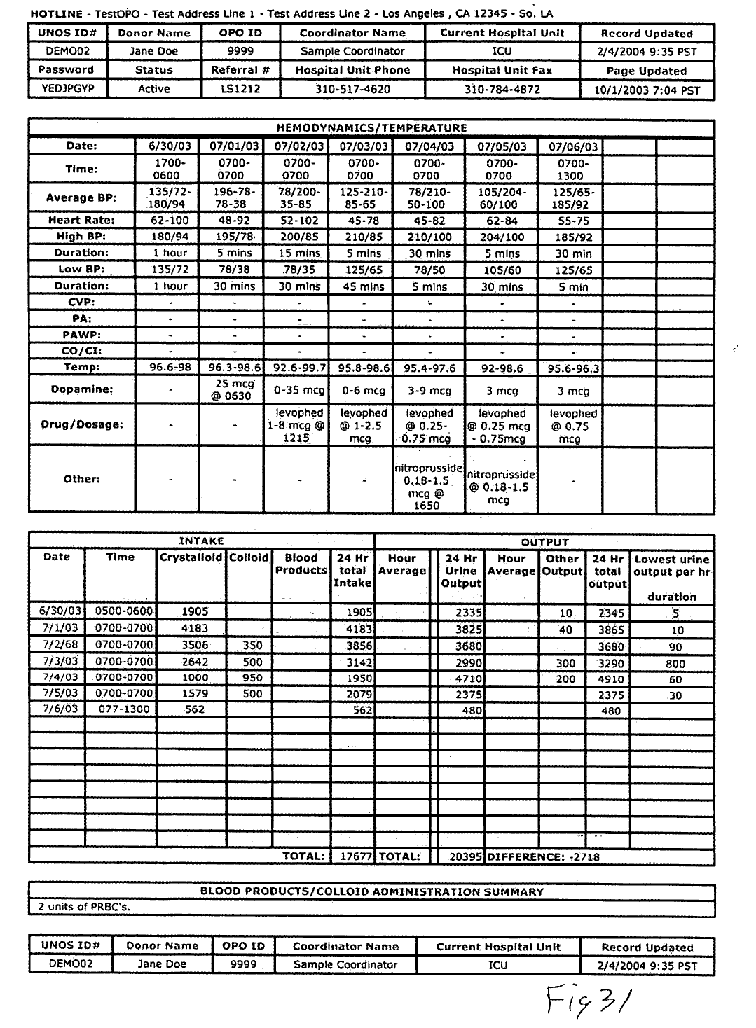

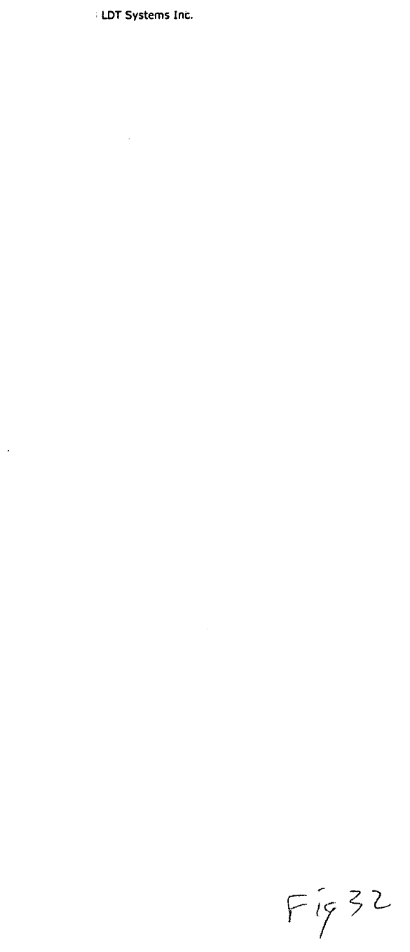

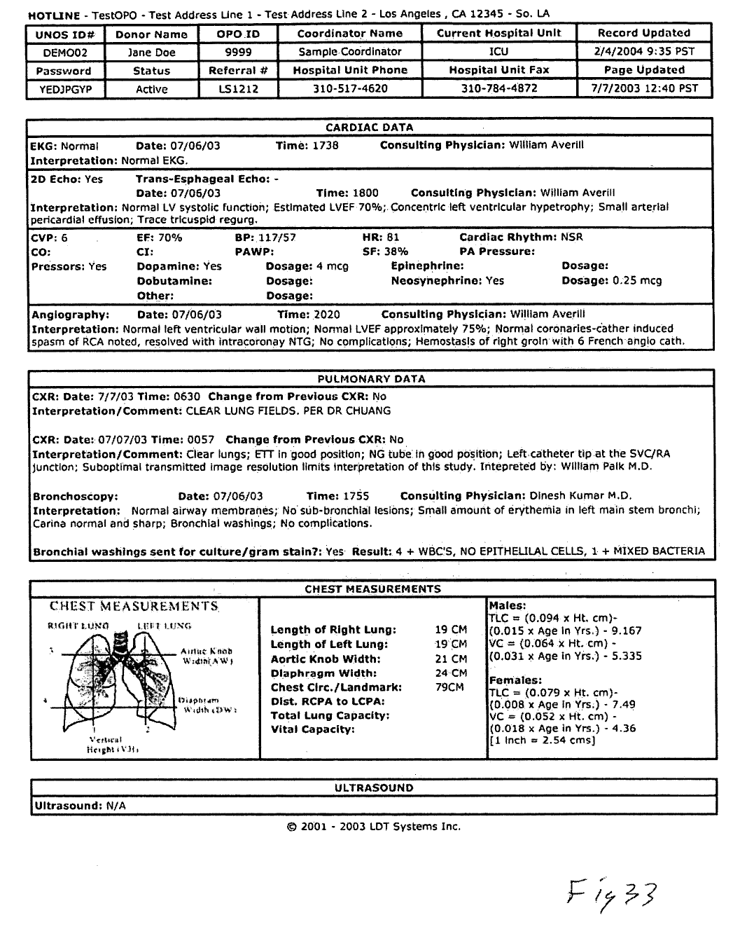

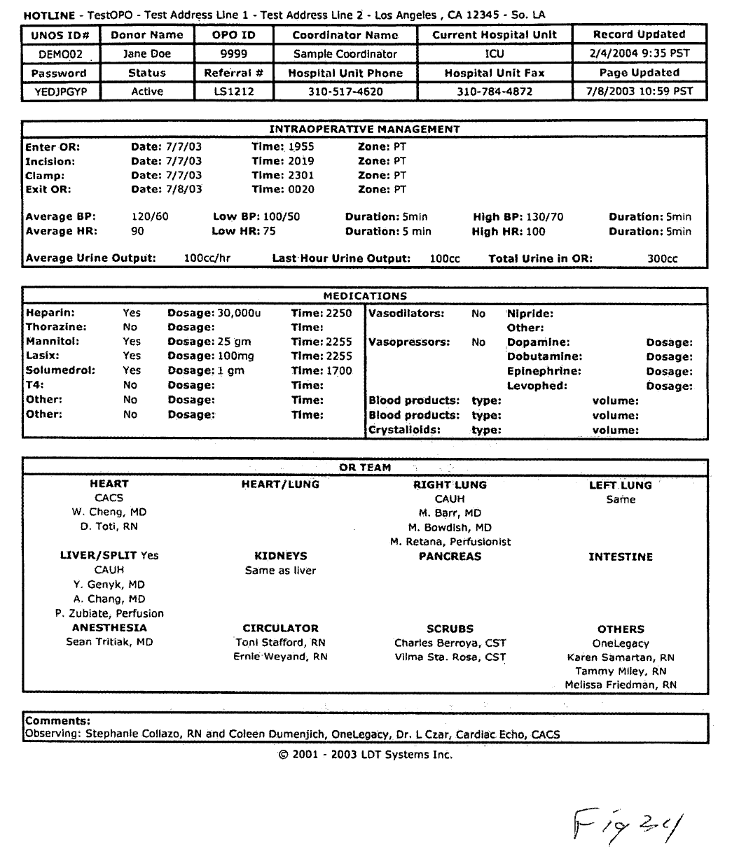

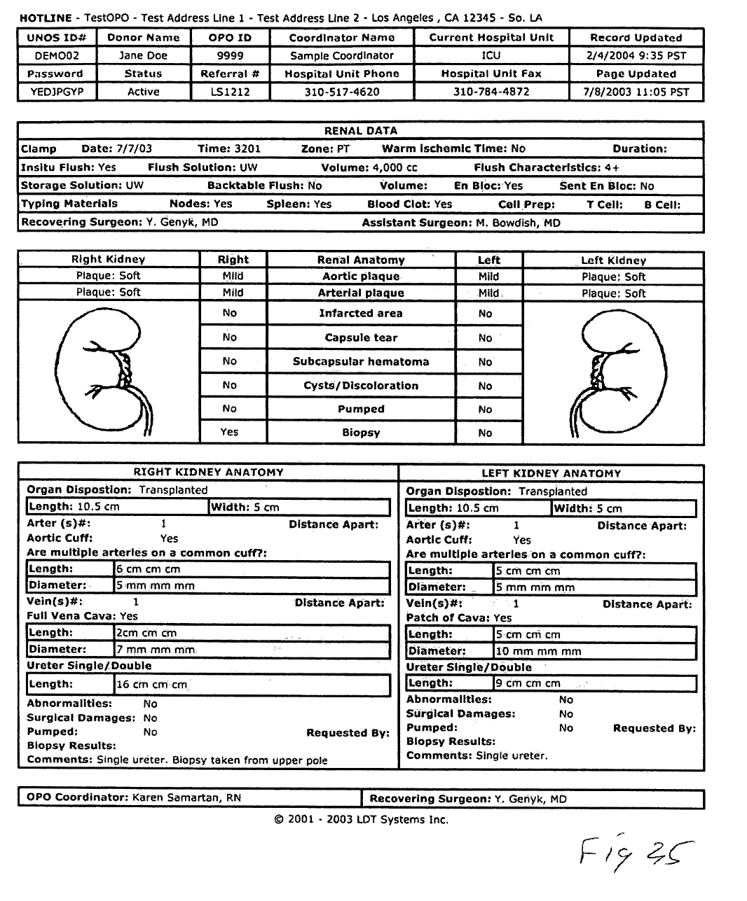

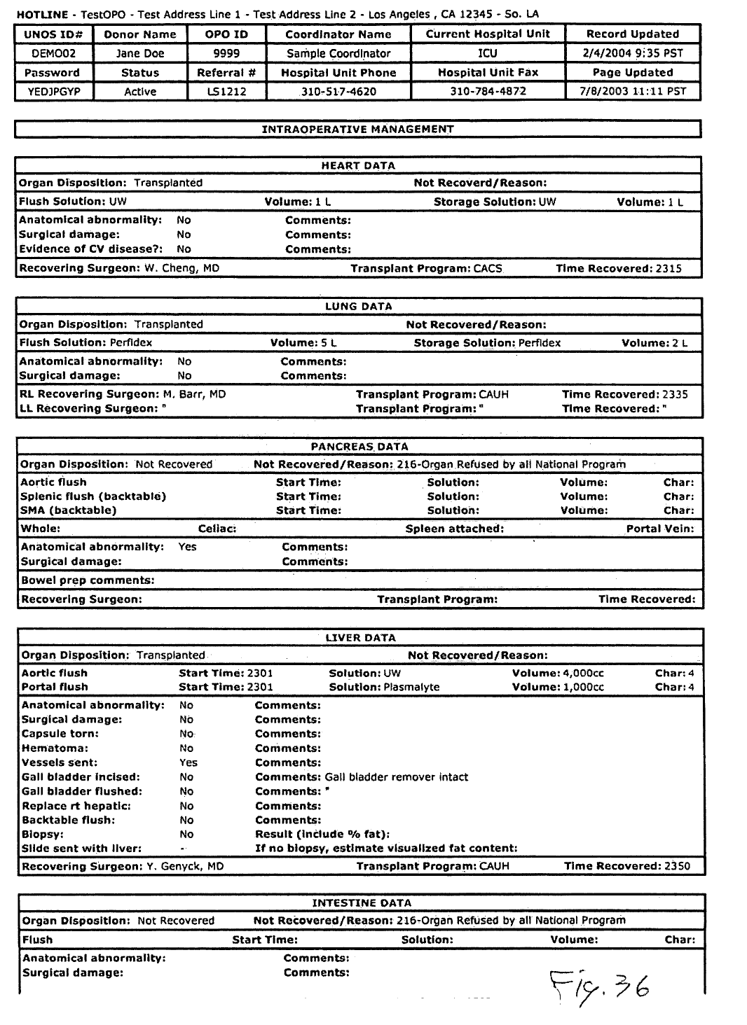

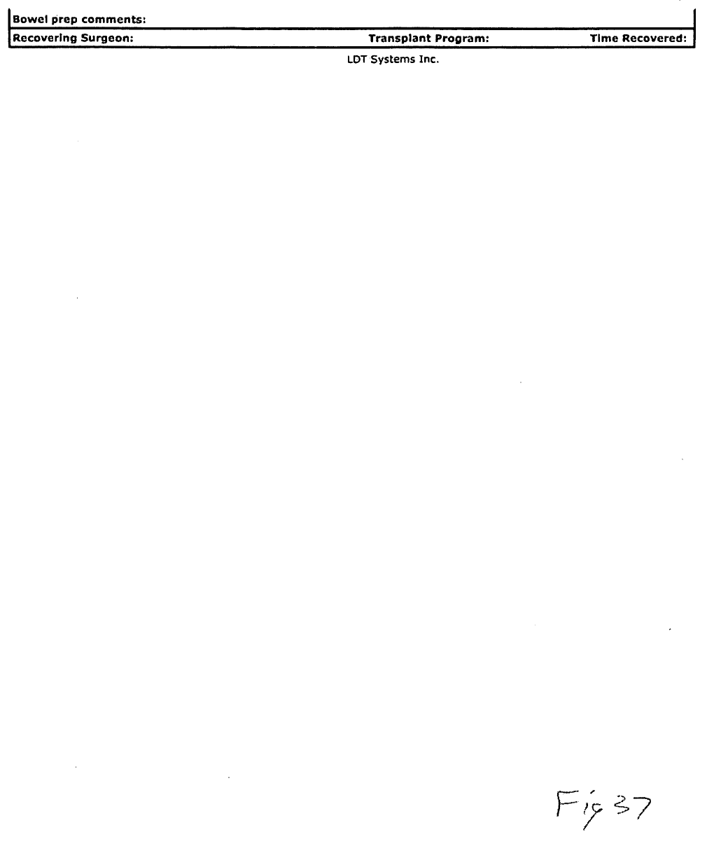

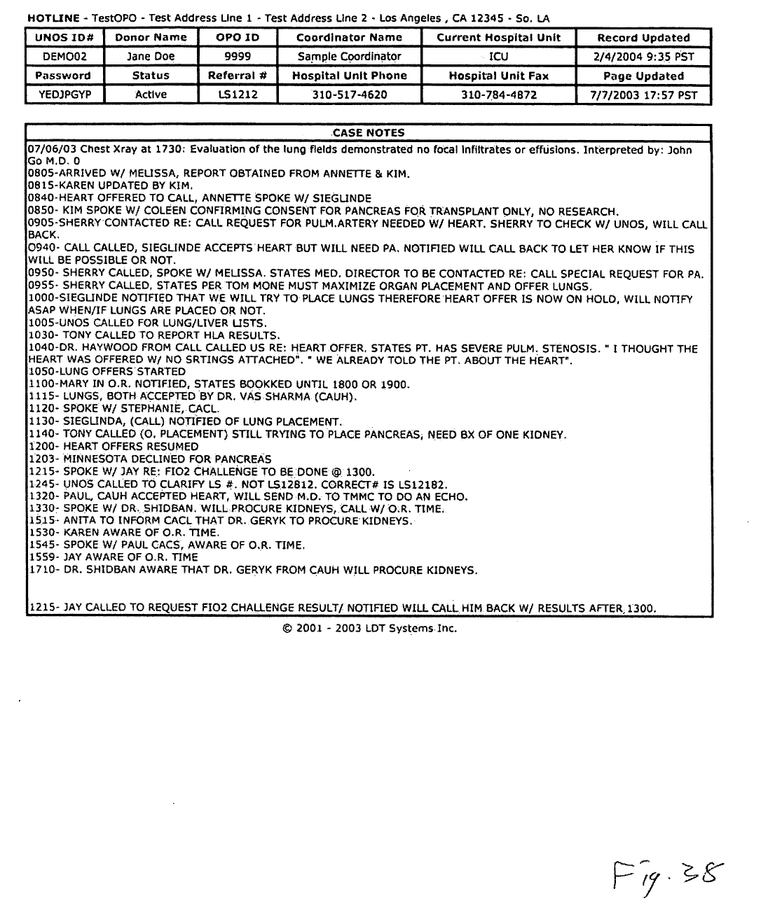

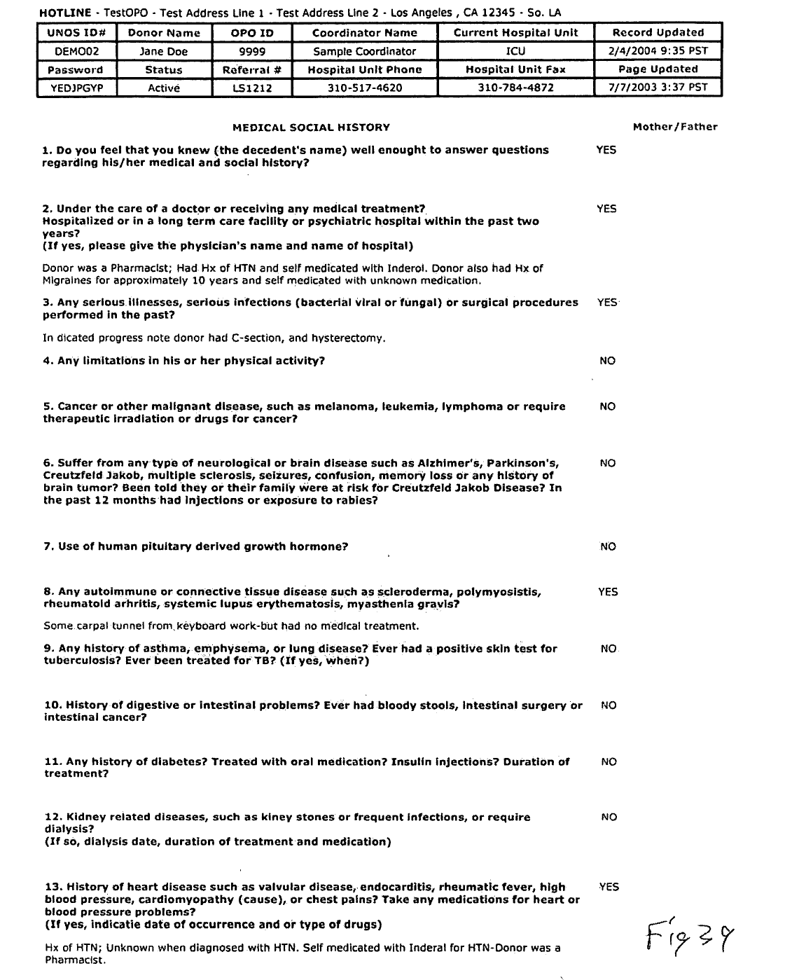

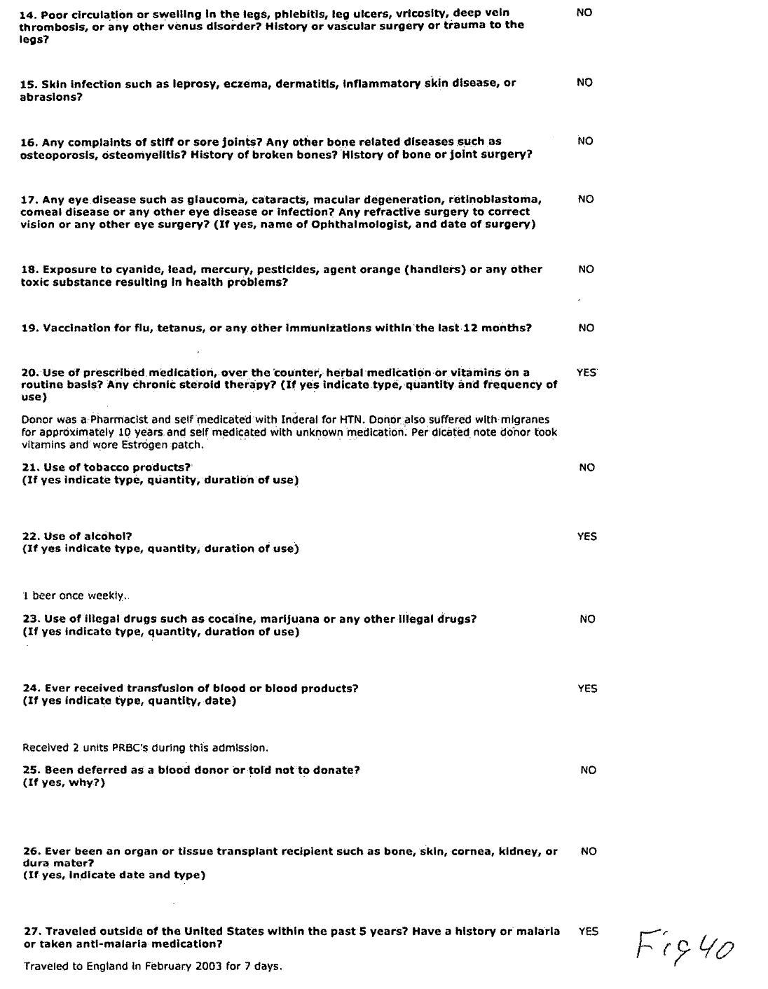

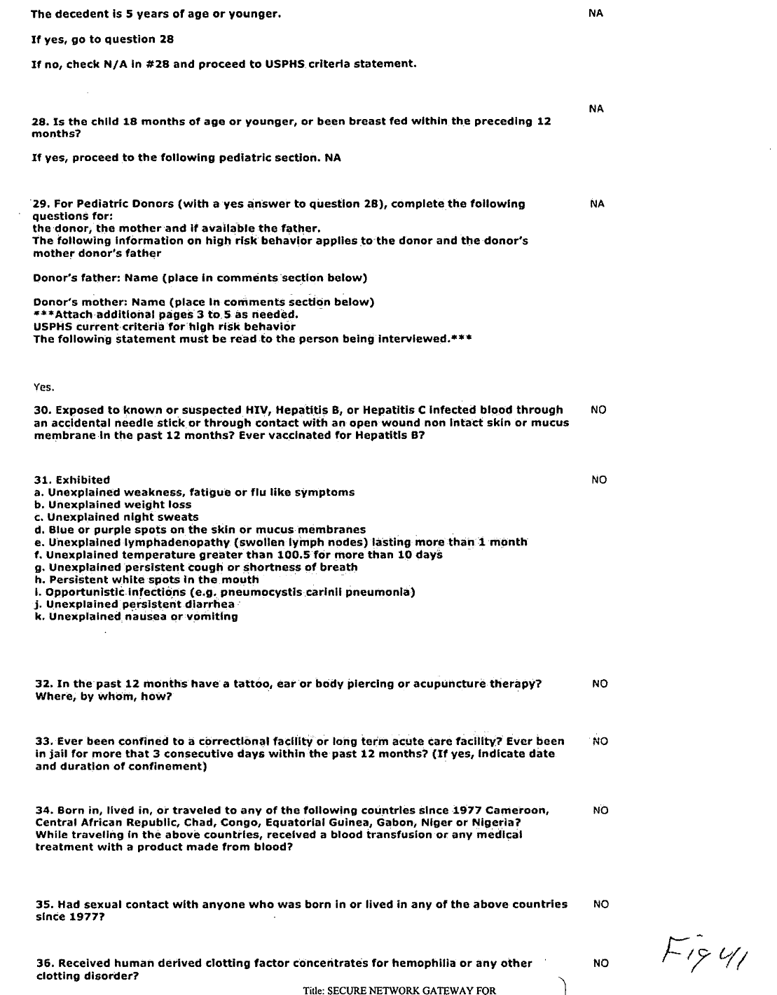

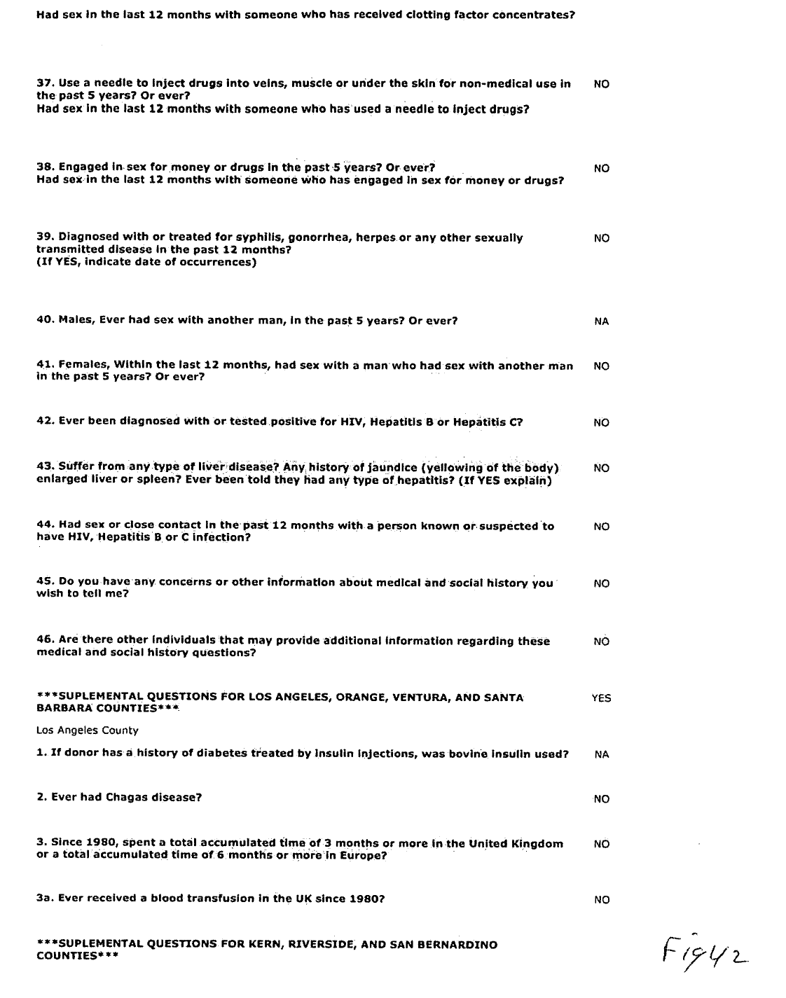

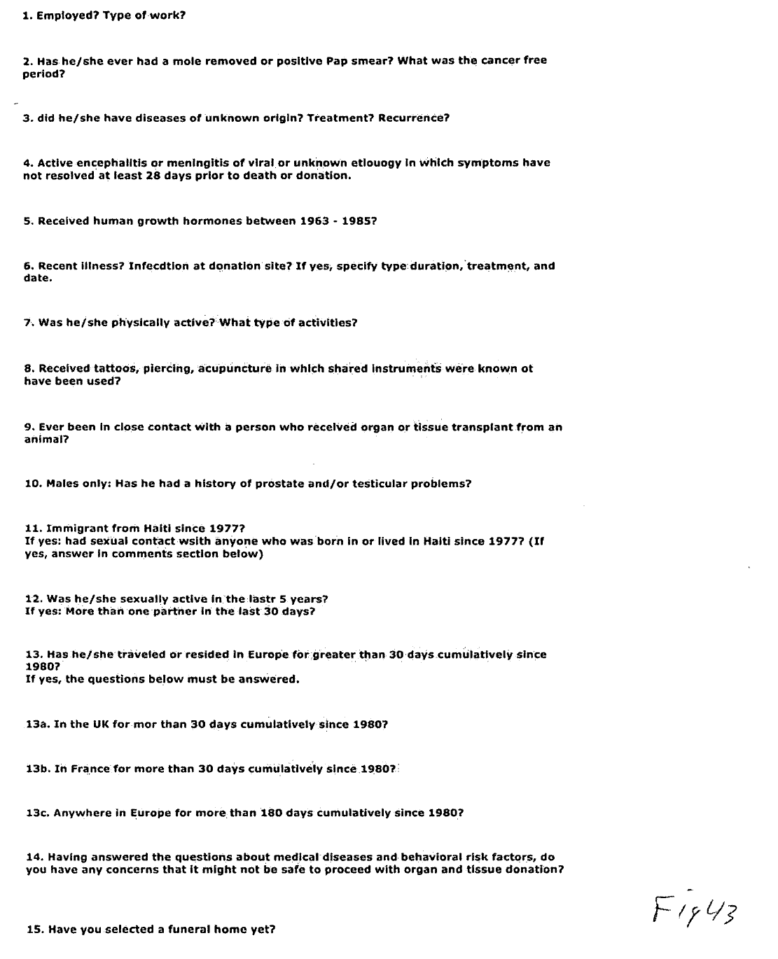

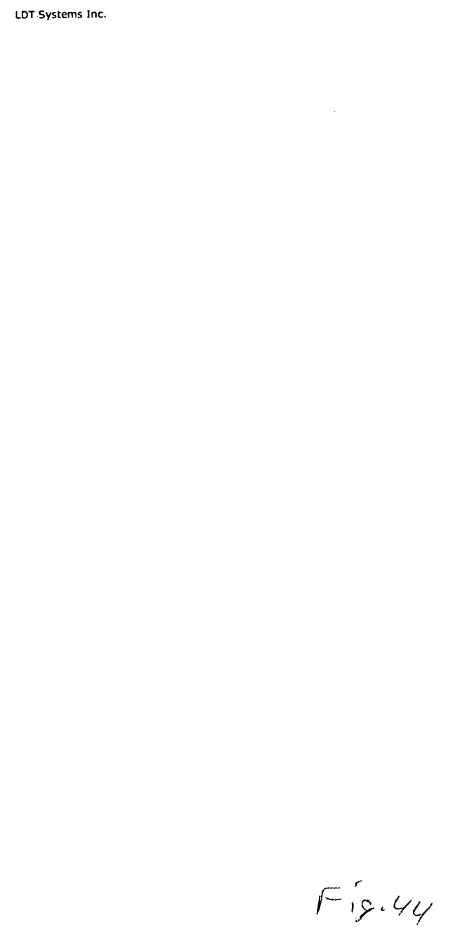

The PTC and/or TPC 20, once consent 24 has been obtained and/or verified, orders a series of tests 26 to be conducted on the donor 16 to identify, among other things, the medical suitability of each potential donatable organ and tissue. In addition to the series of tests conducted at the donor hospital 28, the PTC and/or TPC 20 asks approximately 45 questions on the medical and social health of the donor (drug use, alcohol use, tobacco use, risky sexual behavior, etc.) to compile relevant donor data 30. (See FIGS. 39-44). Exemplary donor data 30 may be in tangible or electronic form and include, without limitation, the donor's emergency room chart, an Intensive Care Unit (ICU) chart, blood type, urine analysis, other lab test results (See FIGS. 18-38), and electronic images such as an EKG, a bronchoscopy, an echocardiogram, a cardiac catheterization, an ultrasound, an X-Ray, and other medical diagnostic imaging and physical examination results.

Referencing FIG. 2, a schematic diagram details an exemplary system architecture for OTRS 10 to facilitate organ and/or tissue donor identification and match notification. OTRS 10 includes an organ and/or tissue data center 42 that: (1) receives organ donor medical data and images electronically (to include facsimile transmissions); (2) allows medical professionals to view donor medical data and/or images electronically; (3) allows OPOs to update donor medical data and/or images; and (4) contains an electronic notification system to alert medical professionals as to the status of a donor case. This status can be, but is not limited to, a new donor case, an updated donor record, an indication that organ placement has begun, an indication that organ placement is ongoing, an indication that organ placement is completed, an indication that an operating room has been scheduled, an indication that organ recovery has begun, an indication that organ recovery has been completed, an indication that tissue recovery has begun, an indication that tissue recovery has been completed. Additional data can be included with the notification to increase the clarity of the notification.

The data center 42 may include a national transplant contact list 46 that may be divided into organ and/or tissue categories and prioritized based in part upon the geographical location of one or more donatable organs and tissue. This list may provide the contact information of registered transplant center representatives and a shift schedule to determine which representatives are available to be contacted based upon their work schedule. The data center 42 may also include an organ and tissue donor database 48 and a donor management log 50. This log may include, without limitation, information on each donor case such as: (1) who accessed the donor chart and when was the donor chart accessed, who made changes to the donor log and when were those changes made; (2) who has accepted one or more organs; and (3) who has recovered and who has transplanted the organs. The donor database 48 may include the relevant donor data 30 as well as other relevant data and images. In addition, the center 42 may include an interface to one or more systems that contain organ recipient data. This interface may allow medical data and images pertaining to the organ donor and the intended organ recipient to be displayed in a comparative manner. Such a comparative display of donor and recipient data increases the speed and improves the organ and/or tissue acceptance process in an attempt to improve the lifespan and quality of life of the recipient of the organ or tissue. The organ and/or tissue acceptance process can be performed manually by a medical professional or can be automated by the OTRS 10 with or without human intervention.

OTRS 10 automates the organ and tissue placement process by providing the PTC 20 with exemplary tools such as, without limitation, a portable computer 56, a wireless phone 58, a wire line phone 59, a wireless PDA 60, a document image scanner 62, a printer 64, wireless network capability 66, wire line network capability 67, medical image management capability 68, a fax machine 69, electronic donor data forms 70, document production software 72, access to a secure gateway 74 that may include the Internet, and any combination of the above.

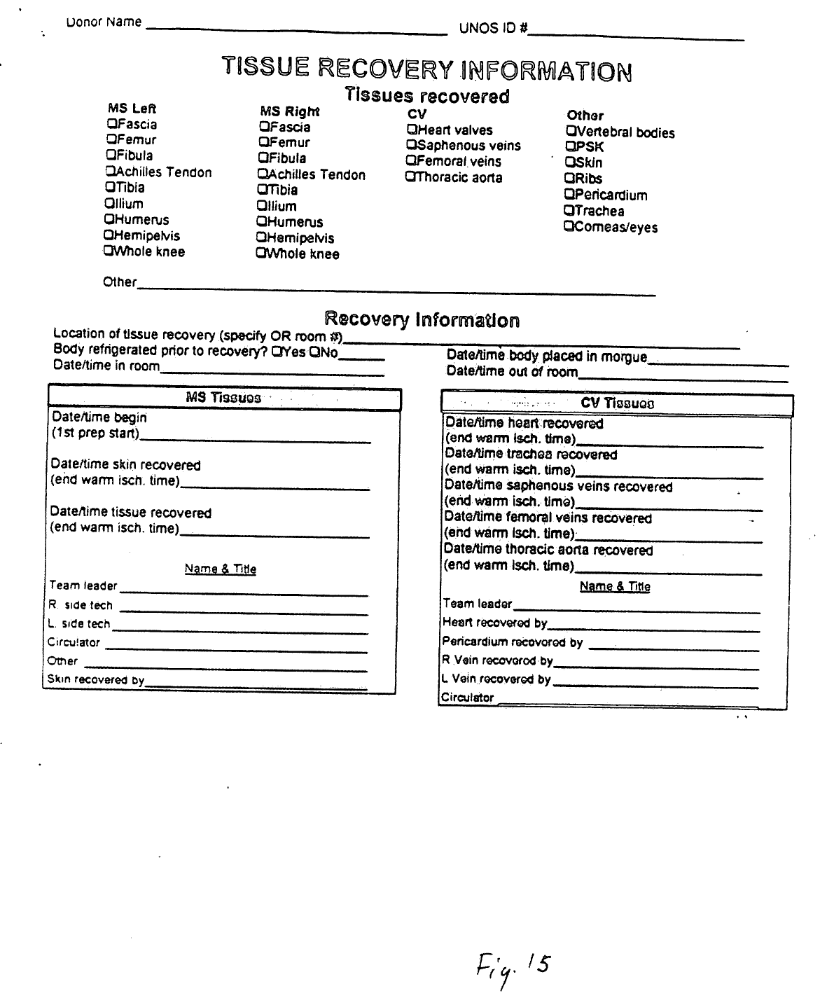

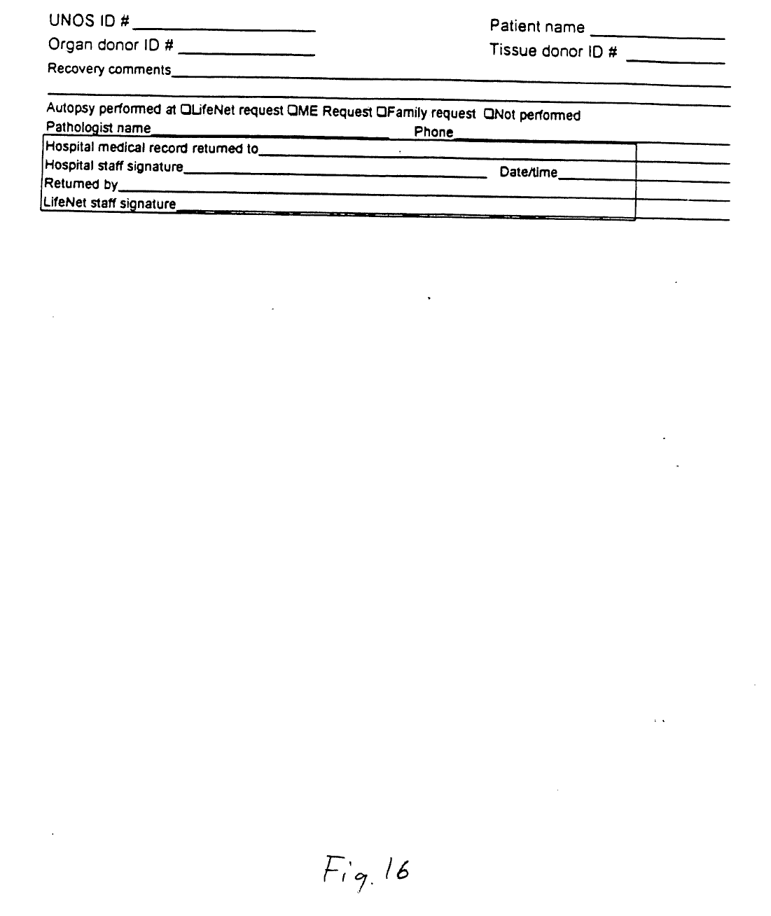

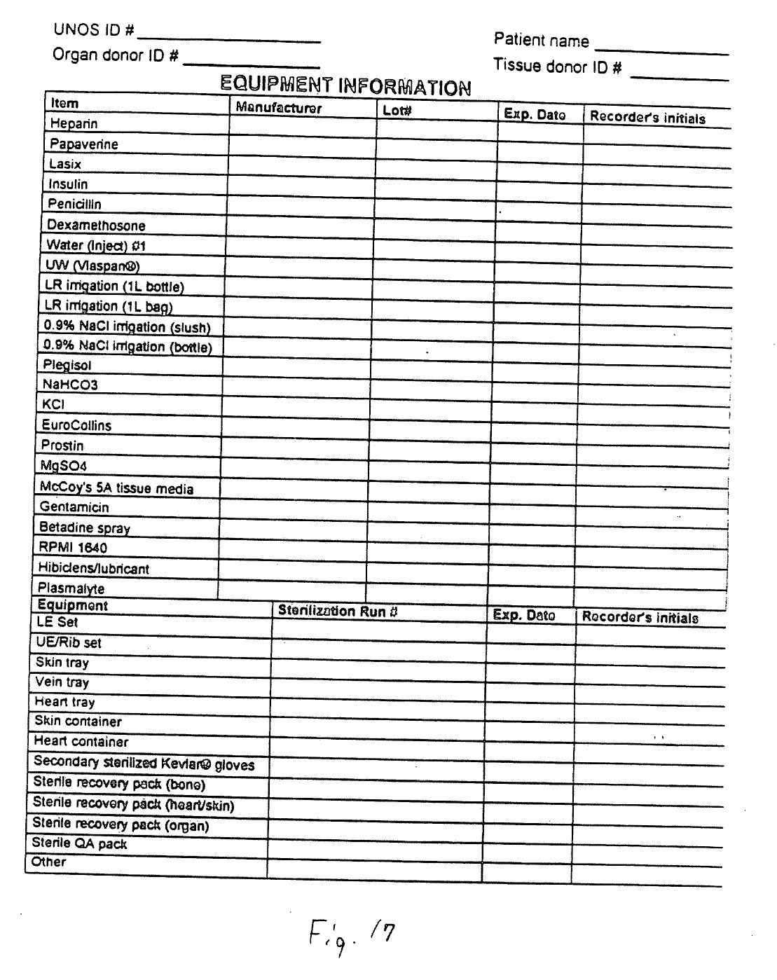

Referencing FIGS. 1-3, the PTC's portable computer 56 may include electronic, fillable versions of donor medical forms 70 (See FIGS. 18-44) that allow the PTC 20 to key in donor data 30 corresponding to one or more predetermined fields 76 (See FIG. 18). In some cases not all of the possible donor data 30 may be easily keyed into the computer 56. Donor data 30 may be converted to an electronic image via an electronic photography or electronic scanning device 62 and electronically attached to the electronic donor form 70. The PTC 20 may also fill out tissue forms (See FIGS. 15-17) if the donor 16 can be a tissue donor. Together, the electronic forms 70, any scanned images, and any electronic attachments comprise the electronic donor chart 78.

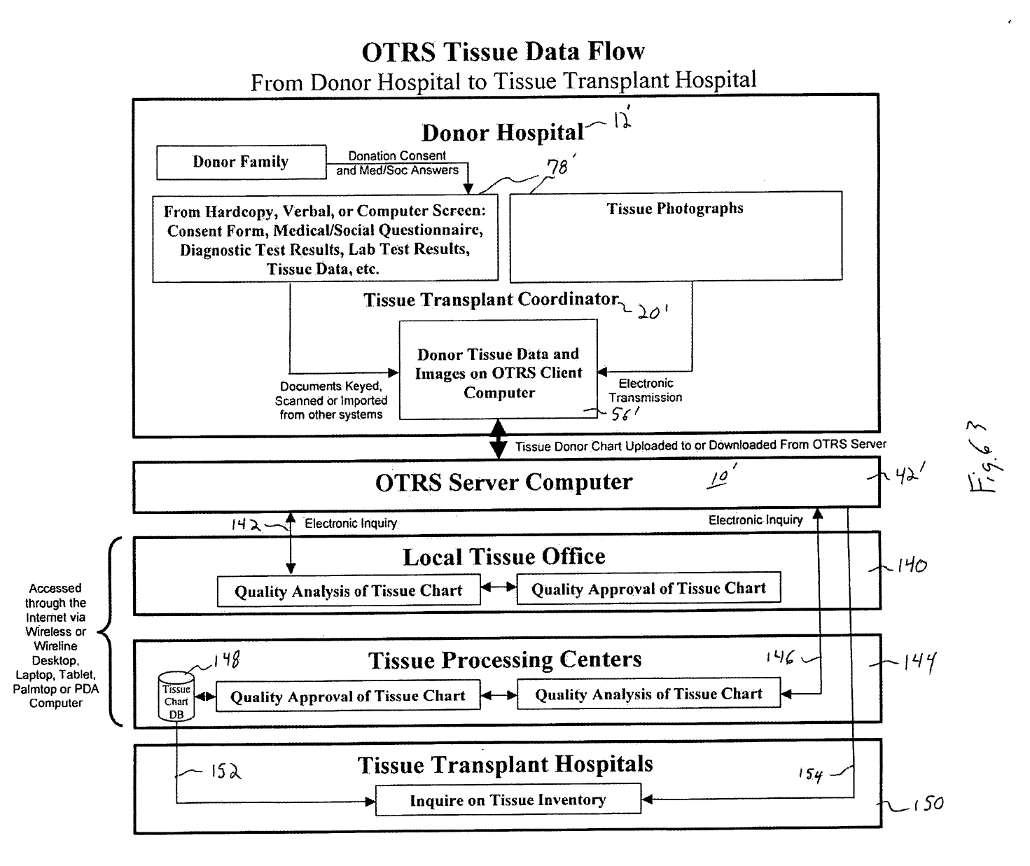

OTRS 10 also has the capability to enable the PTC 20 to attach a variety other forms containing medical information to the electronic donor chart 78. Digital X-rays or digitized photographs of X-rays may be attached to the donor chart 78 and stored and/or transmitted via cable, portable storage device, or via wireless signal. X-rays that are already in an electronic form may simply be transmitted and attached electronically to the donor chart 78. Likewise, video-based images such as, without limitation, echocardiograms, bronchoscopies, cardiac catheterizations, and ultrasounds may be captured digitally and attached to the donor chart 78. The capability of attaching and providing access to electronic images via OTRS better facilitates the rate, quality, and extent of information transfered between donor hospitals 12, transplant centers 80, and tissue processing centers 144 (See FIG. 63).

In a first alternate exemplary embodiment of the present invention, the PTC's portable computer 56 may include electronic printable versions of donor forms 70. The PTC 20 may print out the forms 70 or bring a hardcopy of the some or all of the forms to the donor hospital 12. The PTC 20 may handwrite in donor data 30 upon receiving the results of the labs/tests 28. Thereafter, the PTC 20 may scan the hardcopy forms containing handwritten donor data 30 to create an electronic version/copy of the forms 70. In such an exemplary instance, the electronic scanned copy of the forms 70, along with any other electronic attachments would comprise the electronic donor chart 78.

Upon arriving at the donor hospital 12, the PTC 20 is capable of electronically entering the requisite donor data 30 necessary for transplant centers 80 to discern the likelihood of a match between a donor organ and/or tissue and a potential recipient. The PTC 20 may request that a donor family member 22 respond to questions on the medical and social health history forms 70. A donor family member 22 acknowledges the accuracy of the information inputted regarding the history forms 70 by signing a paper copy of the completed medical and social health history forms 70 or the PTC 20 may capture the signature of the donor family member 22 electronically.

During the donor data 30 entry phase, the PTC 20 may order more labs to better understand the functioning of the donor organ(s). As additional relevant information concerning the medical suitability of the organs and tissue becomes available, the PTC 20 may enter this data into the donor chart 78. The PTC's computer 56 may include a feature where the donor chart 78 is automatically saved on a storage device internal or external to the computer 56 (See FIG. 2) that may be coupled to the computer 56 via a wireless or wireline connection.

Referring to FIGS. 1 and 2, the PTC 20 may connect the computer 56, 84 associated with the electronic donor chart 78 to the secured network gateway 74, which may include the Internet, to upload 79 the donor chart 78 to a central server or data center 42, such as, without limitation, the OTRS server accessible at LDT Systems' Internet site (www.ldtsystems.com). Alternatively, or in addition to, the PTC's computer 56, 84 may be automatically configured to upload the information to the OTRS data center 42 periodically to ensure that a hardware failure associated with the PTC's equipment does not result in complete loss of electronic donor data or the donor chart 78.

Referencing FIGS. 1 and 2, the PTC 20 may connect the computer 56, 84 associated with the electronic donor chart 78 to the secured network gateway 74, which may include the Internet, to download or transfer 79 the donor chart 78 from one remote computer 56, 84 to another remote computer 56, 84 or from a remote computer 56, 84 to a central server and then to another remote computer 56, 84 in order to hand-off the donor case from one PTC to another PTC. Access to the secured network gateway 74 may be provided by a wired or wireless connection, with an exemplary embodiment of the invention incorporating a laptop computer 56 having a wireless modem adapted to access the Internet. OTRS 10 enables the PTC 20 to enter organ and/or tissue donor data into the electronic donor chart 78 while being concurrently detached from or attached to the secure network gateway 74, only to thereafter transmit the donor chart 78 thereover to the central server of the data center 42 in segmented components or all at once as a complete data packet. In an exemplary embodiment, the computer 56, 84 may be preprogrammed to access the LDT Systems' Internet site or the PTC 20 may manually enter the LDT Systems' Internet address to provide a connection therewith. For purposes of explanation only, the present invention will be discussed with the LDT Systems' Internet site facilitating a connection with the OTRS data center 42.

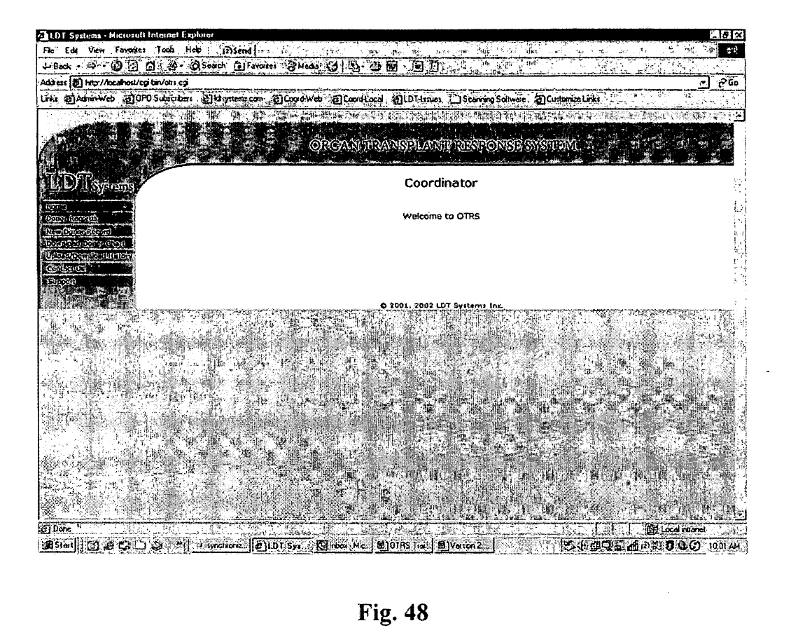

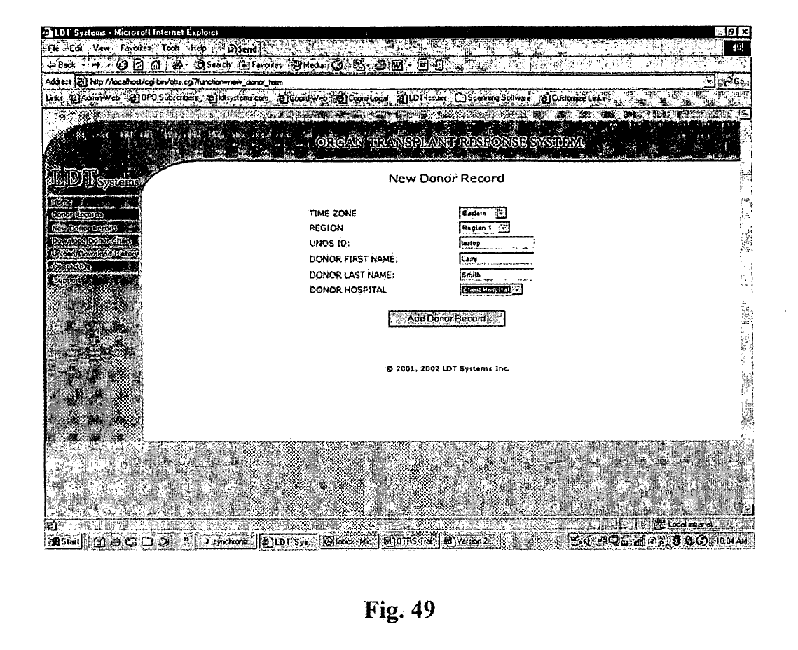

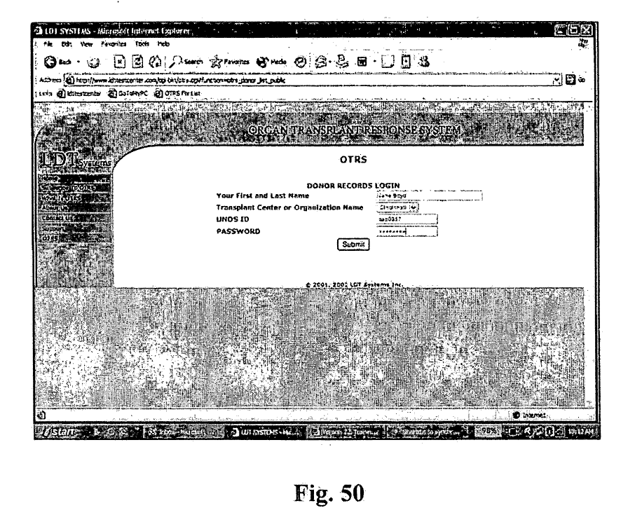

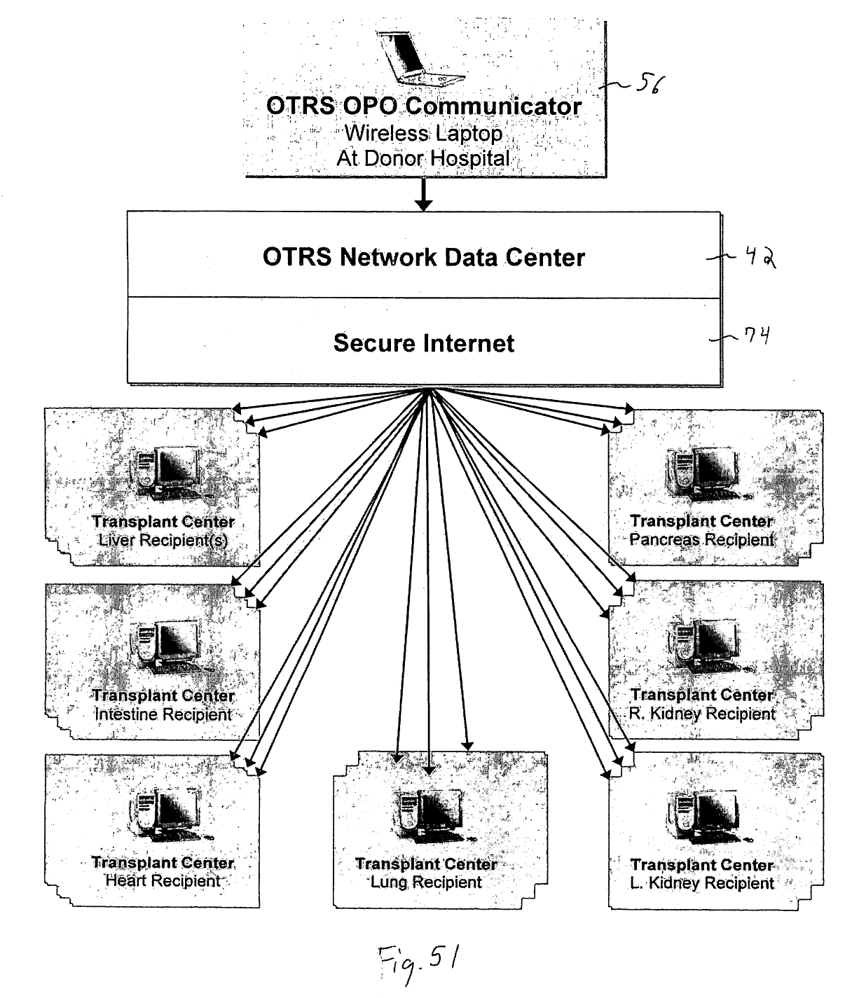

Referencing FIGS. 1 and 47-49, a synchronization process ensues to verify the authenticity of the PTC 20 sender, which may be referred to as a check in/check out process, after the connection via the secured network gateway 74 is established. Subsequent to the synchronization process, the PTC 20 may upload donor data to the OTRS data center 42, such as the donor chart 78 or a component thereof, by accessing the LDT Systems' Internet site. As discussed above, the donor chart 78 may include, without limitation, scanned paper copies such as an EKG or a donor consent form, as well as relevant organ and/or tissue donor data 30. The upload of the donor chart 78 may include uploading the entire donor chart anew or components thereof to replace any preexisting organ and/or tissue donor data 30, or be configured to only upload organ and/or tissue donor data where such fields are blank or no organ and/or tissue donor data 30 is currently entered. It is to be understood that the LDT Systems' Internet site is an exemplary Internet site and that other Internet sites or network data centers may be configured to receive organ and/or tissue donor data 30 and donor charts 78 and organize such data in an analogous manner as performed by the OTRS data center 42.

Referencing FIG. 1, the PTC 20 may make contact with representatives of the Organ Procurement Transplant Network (OPTN) 92 while the donor charts 78 or organ donor data 30 is being uploaded to the data center 42. The PTC 20 indicates to OPTN 92 what organs appear to be transplantable, the medical facility 12 where the donor is located, as well as the age, sex, height, weight and blood type of the donor 16. OPTN 92 will then assign a series of unique identification data for each donor organ associated with a particular donor 16. OPTN 92 maintains a real-time organ-specific list of potential organ recipients that indicates the location of the potential recipients and the medical condition of the potential recipients. Typically, the PTC 20 will ask OPTN 92 to provide data on the potential recipients closest to death that are in closest geographic proximity to the donor (maybe ten pages of results) and another set of potential recipients closest to death nationwide (maybe another ten pages), commonly referred to as a candidate match request 94. OPTN 92 will then provide the PTC 20 with a list of local, regional, and national potential recipients having a need for such organs, with prioritization first going to local potential recipients and then to regional potential recipients and finally to national potential recipients last based on MELD/PELD criteria. Further, the PTC 20 will likely indicate how many pages of each organ list the PTC 20 believes are necessary to place the organ (typically less than twenty-five pages per specific organ). Such an exemplary organ list may include separate lists for placing hearts, lungs, livers, pancreata, kidneys, and intestines. Likewise, OPTN 92 may provide analogous information concerning tissue recipient information.

The requested potential recipient list(s) is transmitted from OPTN 92 to the PTC 20, typically by faxing such list(s) to a machine local to the PTC 20. However, it is within the scope of the present invention that such a requested list(s) is available via a secure network connection, an Internet address, accessible via e-mail, and any means for data transmission known to those of ordinary skill, preferably, but not necessarily secure.

The PTC 20, after having received the OPTN 92 potential recipient list(s) will begin contacting transplant centers 80 associated with prioritized potential organ recipients, such as by telephone. Likewise, it is within the scope of the invention that the PTC 20 notification to transplant centers 80 may include utilization of e-mail or a short message service (SMS) from the PTC's computer 56, 84 and possibly through other computers to one or several computers or mobile computing devices at each transplant center 80 associated with prioritized potential organ recipients. Additionally, such e-mail messages or SMS notifications may contain a link(s) directly to the OTRS data center 42 or LDT Systems' Internet site where the donor chart 78 may be made available to selected third parties, such as intended transplant centers 80 or other OPOs 17.

The PTC 20 will usually attempt to place an available heart and/or lungs prior to placing any other organs such as the liver and pancreas because the time the organs can remain outside the body for hearts and lungs is typically less than other such organs. From the prioritization assigned by OPTN 92, the PTC 20 will contact transplant centers 80 corresponding to potential organ recipients and exchange information with personnel located there, typically a Transplant Coordinator (TC) 96 or a transplant surgeon 104, regarding the available organs. Such exchange of information, commonly referred to as a match request 98, may include a unique OPTN ID (which may be included on the prioritization list supplied by OPTN 92) specific to a particular donor 16, a unique password that may be randomly generated by OTRS 10 for each donor 16, and information regarding accessing the OTRS data center 42, optionally via the LDT Systems' Internet address. The PTC 20 informs the TC 96 that a donor chart 78 and/or other donor data 30 pertaining to a particular donor 16 is available by accessing the OTRS data center 42. To access the OTRS data center 42, the PTC 20 may instruct the TC 96 to enter the LDT Systems' Internet site and logon to the site. Once logged on, the TC 96 may view donor charts 78 and/or other donor data 30 relevant to a particular organ donor 16 and thereafter indicate whether the available organ(s) is accepted for transplant, typically within the requisite one hour from the initial contact with the TC 96.

The PTC 20 may then contact the second priority transplant center 80′ on the OPTN list and conduct an exchange with a TC 96′ at the second transplant center analogous to that discussed above regarding the first priority transplant center 80. This process is repeated until the transplantable organ(s) is accepted by a transplant center 80, 80′. Prior art methods of exchanging information regarding any available organ between the PTC 20 and the TC 96 averaged over twenty minutes per TC 96 contacted, in large part because the exchange of information was limited to telephonic exchange of voice data. OTRS 10 has reduced the contact time between the PTC 20 and the TC 96 to approximately three minutes. This is particularly important considering that an average number of contacts per donor 16 is thirty-five. Therefore, total placement time on average is typically two hours using OTRS 10, whereas the prior art method and systems took upwards of twelve hours per donor 16.

Referring to FIGS. 1, 21, and 22, the TC 96 in attempting to access the OTRS data center 42 via the LDT Systems' Internet site is instructed to logon to the LDT Systems' Internet site by inputting one or more pieces of relevant information. Such relevant information may include, without limitation, a donor ID, a unique password supplied via the correspondence with the PTC 20, a TC 96 identifier, and a transplant center 80 identifier. Upon accessing the OTRS data center 42, each TC 96 may gain access 100 to the electronic donor charts 78 and/or donor data 30 for the potential donor(s) 16 in question. It is within the scope of the present invention that the access 100 provided to the TC 96 be limited to donor charts 78 and/or donor data 30 for the potential donor(s) 16 in question.

After a transplant center 80 has accessed a donor chart 78 and/or donor data 30, a decision to accept or decline the offered organ(s) is submitted, as evidenced in an exemplary form, by noting such acceptance or decline (based on a priority basis) on OTRS 10 in approximately real-time. It is generally understood that the acceptance of an organ will be most likely indicated directly by the transplant surgeon 104 in charge of caring for the potential organ recipient, however, it is within the scope of the invention that such acceptance comes from an OPO 17 serving the transplant center 80, a hospital administrator 106 from the transplant center 80, or the TC 96 of the transplant center 80. The transplant center 80 personnel may contact the transplant surgeon 104 by phone and read off the donor chart 78 and/or donor data 30 along with relevant potential organ recipient data. If the transplant surgeon 104 has access to the Internet, the surgeon may access LDT Systems' Internet site to view the electronic donor chart 78 and/or donor data 30 to make a decision to accept or decline the available organ(s). In an exemplary embodiment, the surgeon 104 is equipped with a Web-phone and can access the Internet wherever cellular communications are enabled to review the donor chart 78 and/or donor data 30. The transplant surgeon 104 may also request that additional tests be conducted such as, without limitation, biopsies, additional X-rays, and/or bronchoscopies on the potential transplantable organ(s) prior to any acceptance of the organs.

Each transplant center 80 contacted by the PTC 20 is given one-hour to respond with verification of a match 102. In some instances, the PTC 20 will continue to make contacts with other transplant centers 80′ just in case the first offered transplant center 80 refuses the organ offer. If no local transplant centers 80, 80′ accept one or more of the available organs, the process of contacting transplant centers may continue for transplant centers associated with regional potential organ recipients, and finally may end with transplant centers associated with national potential organ recipients if any unplaced organs remain. International organ offers are made on occasion when the recipient list has been exhausted.

The PTC 20 may telephone a non-local OPO representing those non-local transplant center(s) who represent or are associated with a potential organ recipient and provide the requisite information to enable the non-local OPO to access one or more donor charts 78 and/or donor data 30. The process by which the non-local OPO accesses the donor charts 78 and/or donor data 30 may be the same as discussed above for the transplant centers 80, 80′, with each OPO and/or transplant center using an Internet-capable computer to logon to the LDT Systems' Internet site and access the OTRS data center 42.

Once the organs have been accepted, recovery thereof begins. However, under certain instances, not all of the transplantable organs will be placed prior to recovery. In certain instances, a transplant surgeon 104 may recover the organs or have a local surgeon conduct this part of the procedure, which takes typically less than one hour to recover all of the transplantable organs. Again, time is critical and if the time elapsed becomes too great, the transplant surgeon 104 may request that the local surgeon conduct the removal of the organs and transport the organs to the awaiting transplant surgeon. Even as the recovery is taking place, more donor data 30 may be entered, usually by the PTC 20, and thereafter uploaded to the OTRS data center 42. The PTC 20 may leave the donor hospital 12 after entering and uploading any last minute information onto the OTRS data center 42.

After recovery, each organ is placed in a cooler following prescribed preservation protocols along with copies of relevant organ and donor data 30. Exemplary forms of relevant organ and donor data 30 include tangible copies organ measurements, recovery team names, and the donor chart 78. Each cooler may also contain two vials of the donor's blood for type-testing at the transplant center 80, 80′.

In instances where the organ recovery team will likewise conduct the transplant surgery for the intended organ recipient, the recovery/transplant team returns to the transplant center with the organs for transplant. While the recovery is taking place, a second team at the transplant center is preparing the intended organ recipient for transplant surgery, which typically involves correlating the surgery with the intended arrival time of the recovery/transplant team. In a preferred instance, the defective organ is removed from the intended recipient just as the transplantable organ and the recovery/transplant team arrive at the transplant center. Using the present invention, the transplantable organ is prepared for transplant and transplanted without further loss of time or increase in the cold ischemia time.

Any time during which the organ is outside the body, cold ischemia time, damages the organ. Adult hearts can last typically up to 4 hours outside a body. Adult kidneys can typically last 24-48 hours outside the body. This is why typically 90% of all kidneys are transplanted, but typically only 35% of all heart are transplanted.

As discussed above, OTRS 10 is directed to a network-based information system enabling the donor chart 78 and/or donor data 30 to be entered locally onto a computer 56, 84 and uploaded onto the OTRS data center 42 and later retrieved by privileged personnel, such as an intended transplant center 80. In a more detailed exemplary embodiment, the secure network gateway 74 encompasses the data center 42, which includes the organ donor database 48, and the Internet or private network. The local OPOs 17, transplant centers 80, and authorized personnel of such may utilize an OTRS local application that may include the same or similar code and database structure as the OTRS server application. An exemplary code is Hyper-Text Marked-up Language (HTML) and an exemplary database structure is Structured Query Language (SQL).

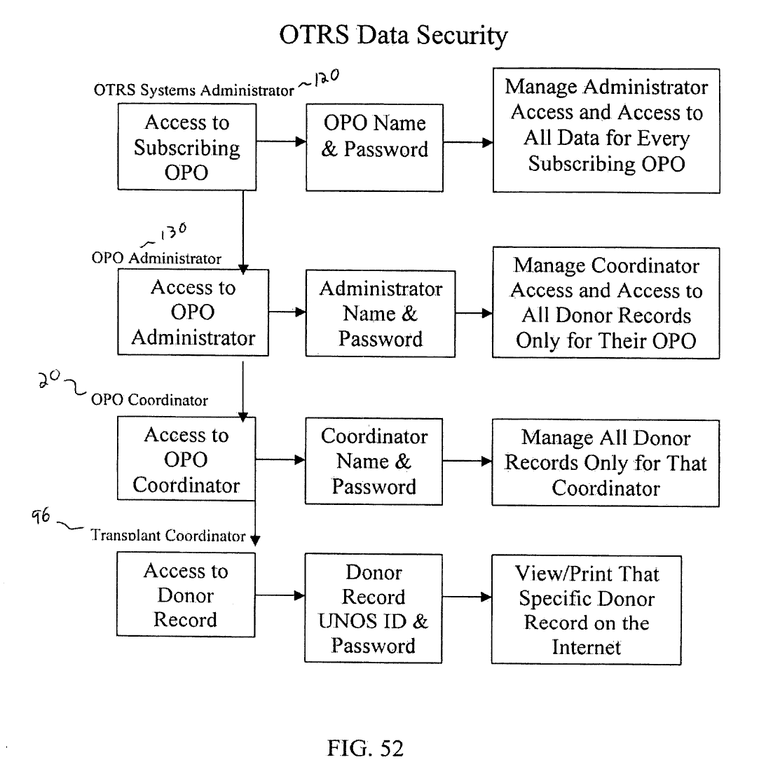

Referencing FIGS. 1 and 52, donor data 30 is generated at the donor hospital 12 and thereafter uploaded to the OTRS data center 42. The donor data 30 is made available in a preferred embodiment via the Internet to specific transplant centers 80, 80′ and may be made available to specific OPOs for determinations as to acceptance or decline of one or more available organs. Within the OTRS communication process is an embedded hierarchy and accessibility to the OTRS data center 42 that may include at least four general groups: OTRS systems administrators 120, OPO administrators 122, PTCs 20, and TCs 96.

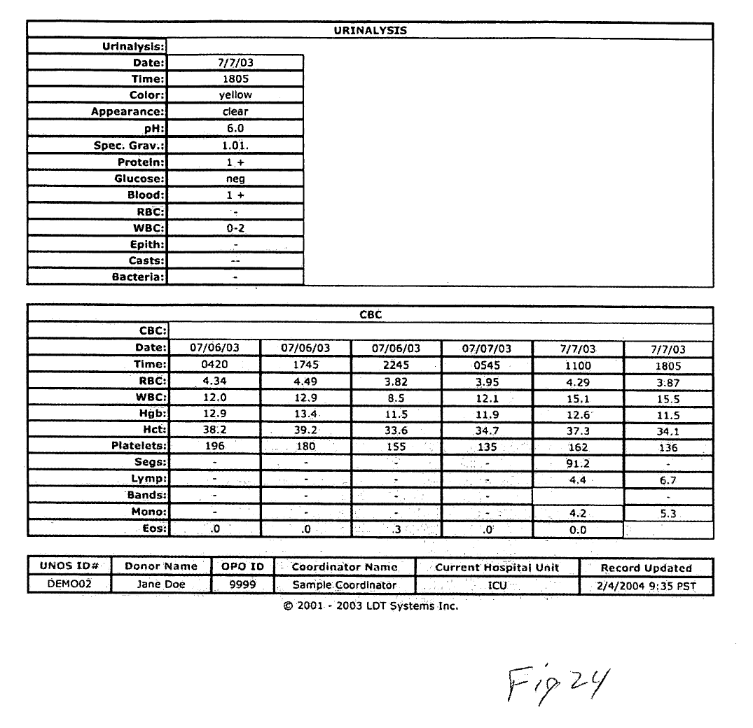

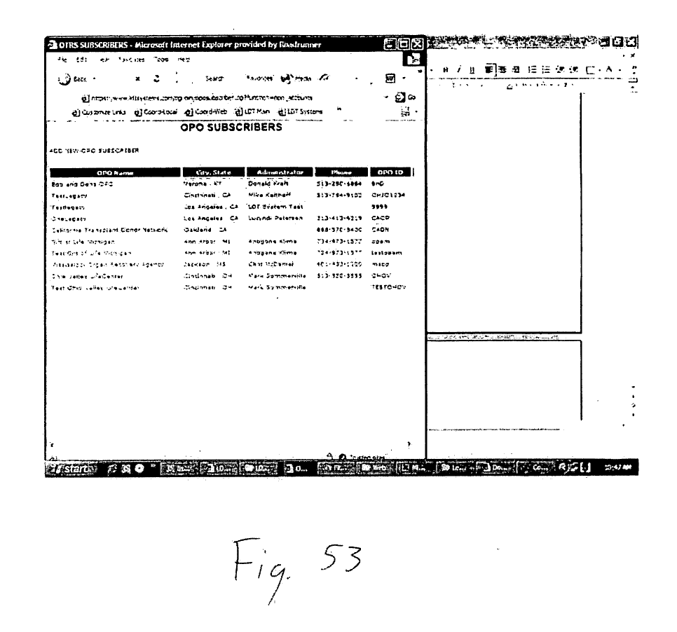

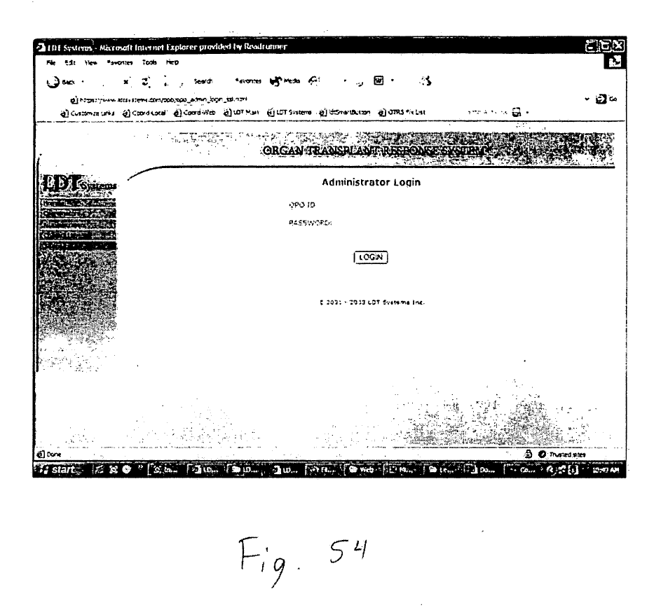

Referring FIGS. 52 and 53, the OTRS systems administrators 120 oversee the OPO accounts and ensure access is provided to authorized OPOs based upon subscription to the services provided. For each subscribing OPO, a record is entered into the OTRS application by the OTRS systems administrators that may include, for example, without limitation, the OPO name, an OPO identifier, the OPO address, OPO Administrator(s) name and password, and OPO contact information such as telephone numbers and facsimile numbers. Once the OPO data is input into the system, the OTRS application is operational and ready for OPO data input and/or extraction/viewing of data. Generally, the OPO subscriber listings, as shown in FIG. 24, are viewable only by the OTRS systems administrators 120. OTRS systems administrators 120 also ensure that OPO subscribers are provided with a secure network connection, manage the OPO administrator 130 access, and update in virtually real-time donor data 30 uploaded and confirmed by the OPO administrators, and in some cases, PTCs 20. Both OTRS applications (local & Internet) include a one-time OPO set-up program conducted by the OTRS systems administrators 120.

Referencing FIGS. 52 and 53, the OTRS software itself provides a tiered access approach, with the OPO administrator 130 managing PTC 20 access, was well as access to all donor records specific to that OPO. An OPO administrator 130 will sign onto the OTRS application to locally setup OTRS for that particular OPO using a unique identifier such as their first name and/or last name and password. This unique OPO administrator identifier must match the data entered by the OTRS systems administrators 120 before the OPO administrator 130 features to be available via the software application. A unique password and user identifier are examples of security measures that may be incorporated into the OTRS software for security reasons for each OPO administrator 130 and each PTC 20 associated with the OPO. Thus, if a user's unique identifier and OPO name are in the OPO subscriber record as an OPO administrator 130, then “Admin” will be automatically attached to the user's record.

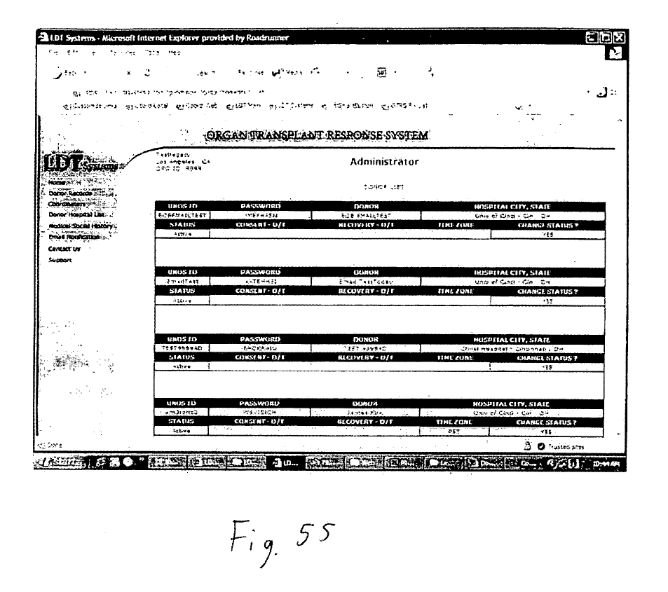

Referring to FIGS. 52 and 55, the OPO administrator 130 generally will be given rights to add, change, and delete PTC 20 records. Such PTC 20 records may include the first and last name of each PTC 20, the PTC's 20 status, the PTC's 20 password, the OPO-specific ID, and PTC 20 contact information that might include office, cellular, and pager numbers as well as text messaging and e-mail addresses. The local application may use the same file to store OPO administrator 130 and PTC 20 records. OPO administrators 130 may be responsible for adding each PTC 20 or other user (surgeon 104, for example) to their OPO list of authorized users. Further, the OPO administrator 130 may change the status of a donor chart 78 from “inactive” to “active” or vice versa. This process will confirm that all data for that record is residing on the OTRS data center 42. Once confirmation is complete, the status of that record 78 will be changed to “inactive”, but will not function to delete that record. “Inactive” status eliminates the ability of a PTC 20 to change the data 30 associated with a donor record 78, but it still allows the record to be viewed by the permitted staff of the originating OPO. “Inactive” status also disables a transplant centers' 80 or non-local OPO's access to the donor record 78, even if the transplant center 80 or non-local OPO has the correct OPTN ID and password for the particular donor record 78.

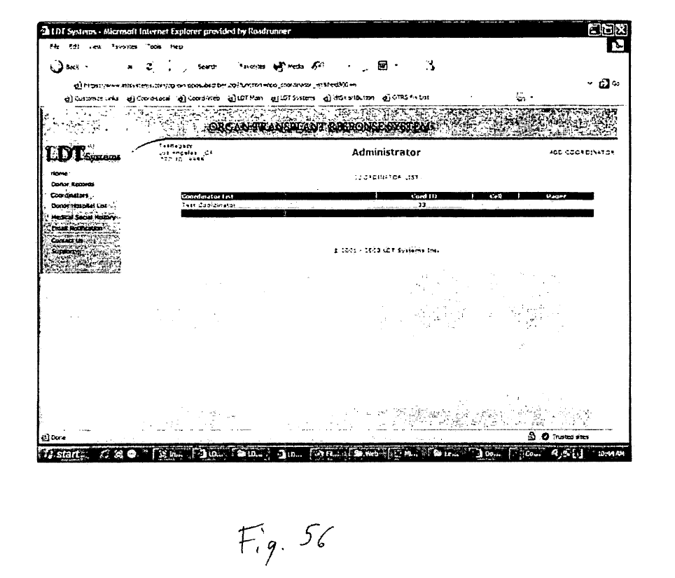

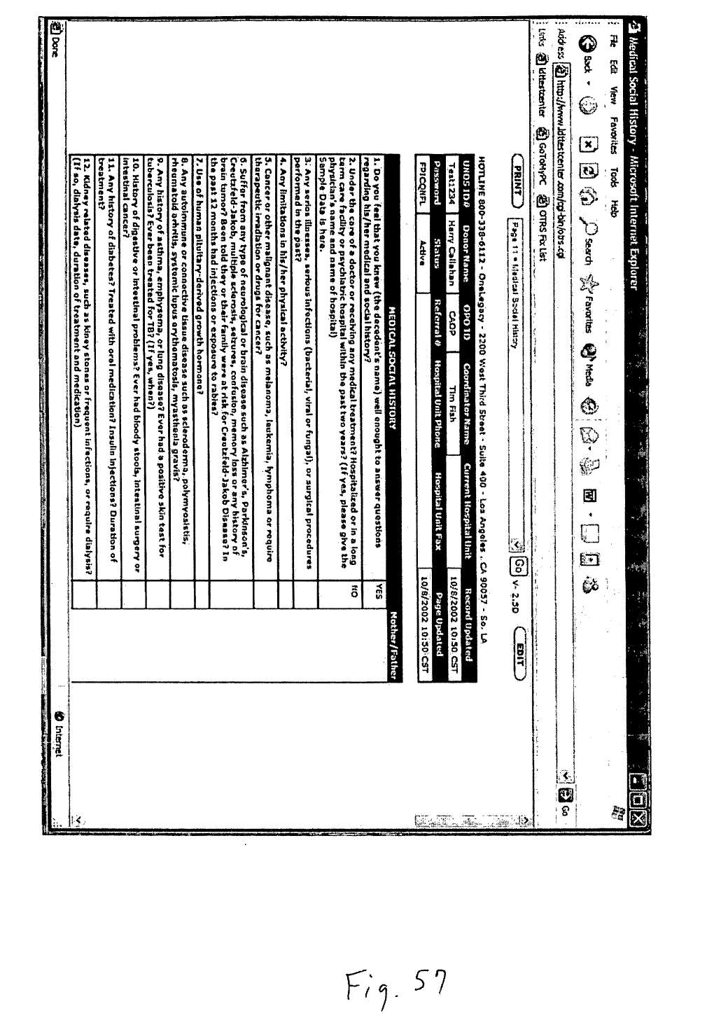

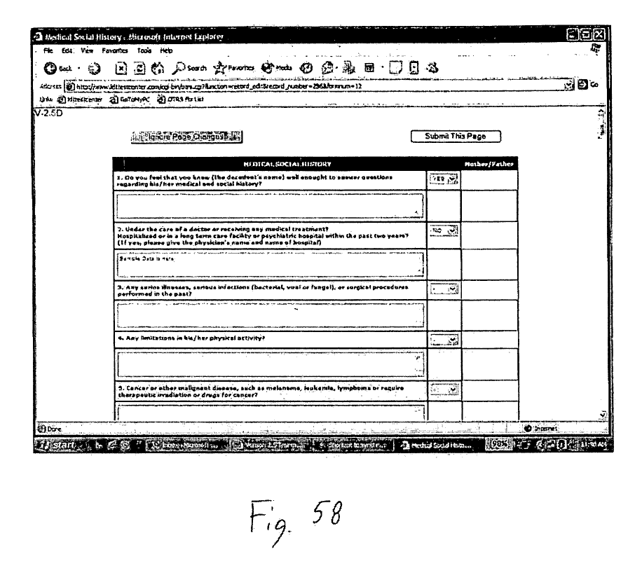

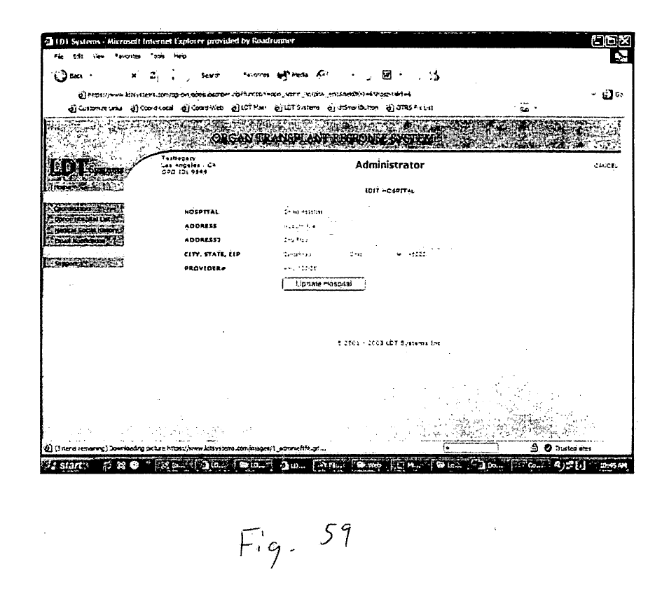

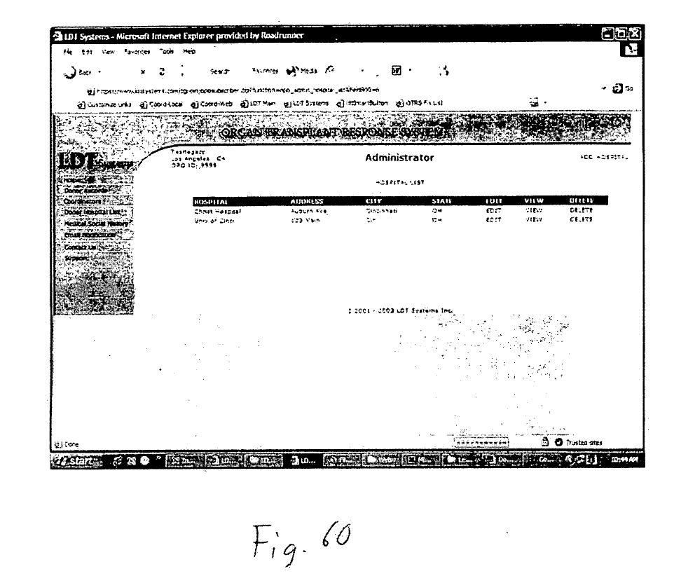

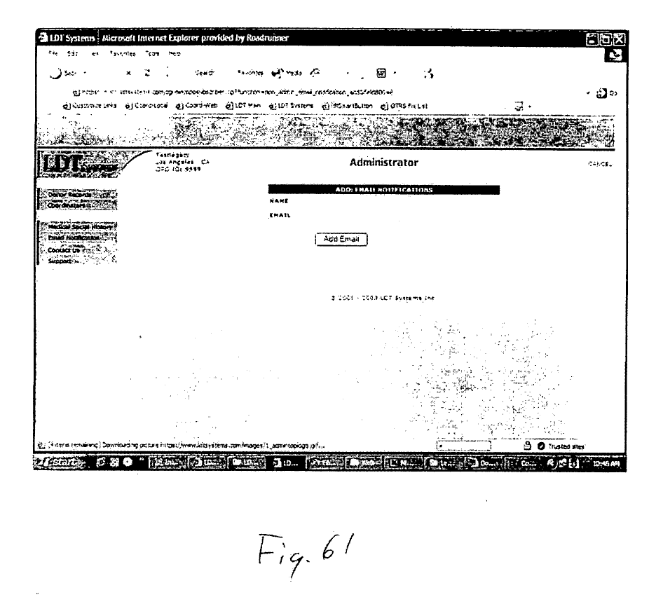

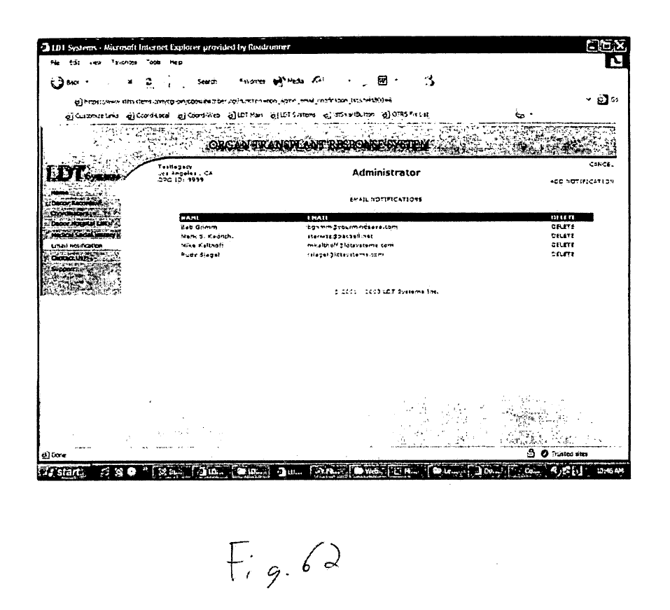

Referencing FIGS. 56-62, the OPO administrator 130 may also have the ability to add or delete PTCs 20 from the system (See FIG. 56), as well as amend some of the medical and social history questions asked of each donor 16 (See FIG. 57) and review the questions as shown (to be viewed by each PTC 20) in amended form (See FIG. 58). Further the OPO administrator 130 may be responsible for inputting and updating information on each donor hospital 12 that is associated with that particular OPO (See FIG. 59), as well as reviewing and updating a listing of all donor hospitals 12 associated with the OPO (See FIG. 60). The OPO administrator 130 may also be responsible for inputting and updating the names and e-mail address of those individuals that are to receive e-mail notification of a new donor listing being uploaded to the OTRS data center 42 (See FIG. 61) as well as verifying that the summary listing includes those individuals intended to receive such e-mail notification (See FIG. 62).

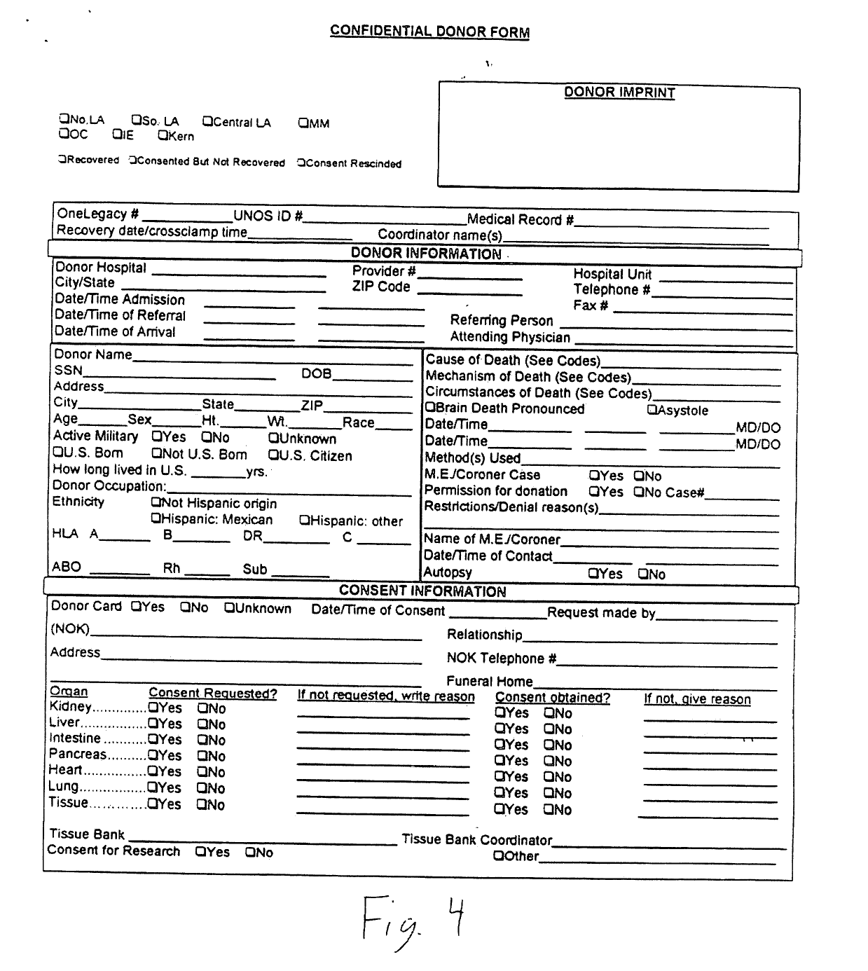

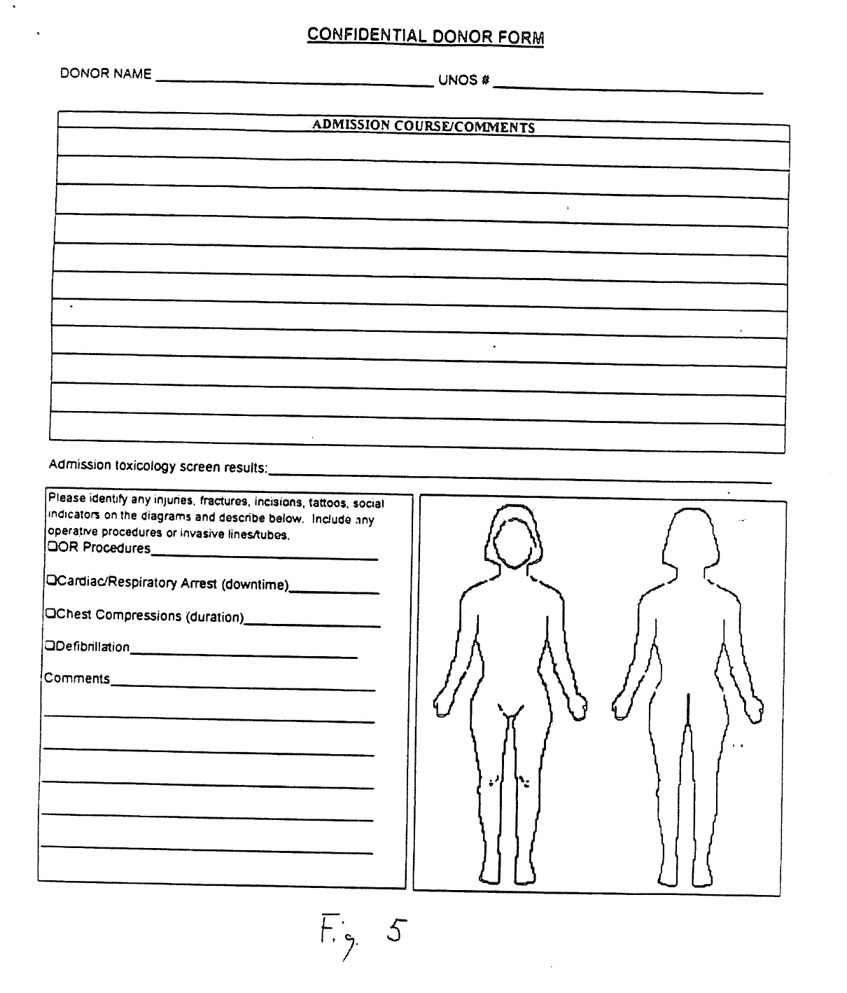

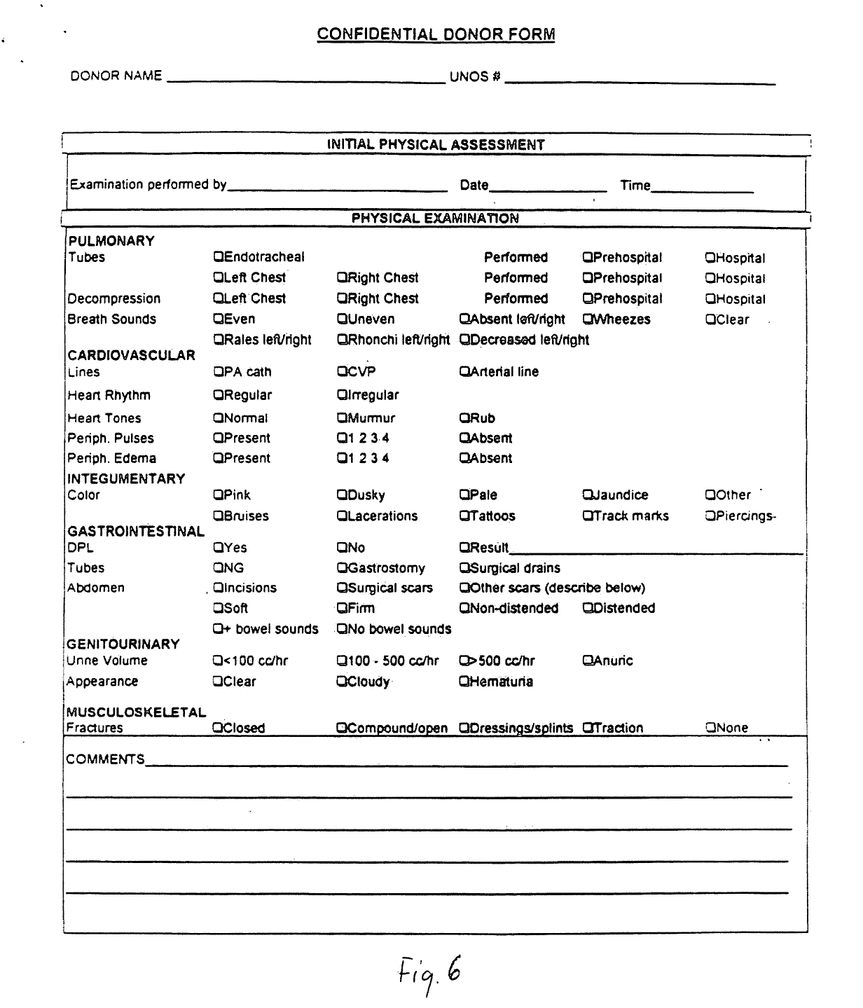

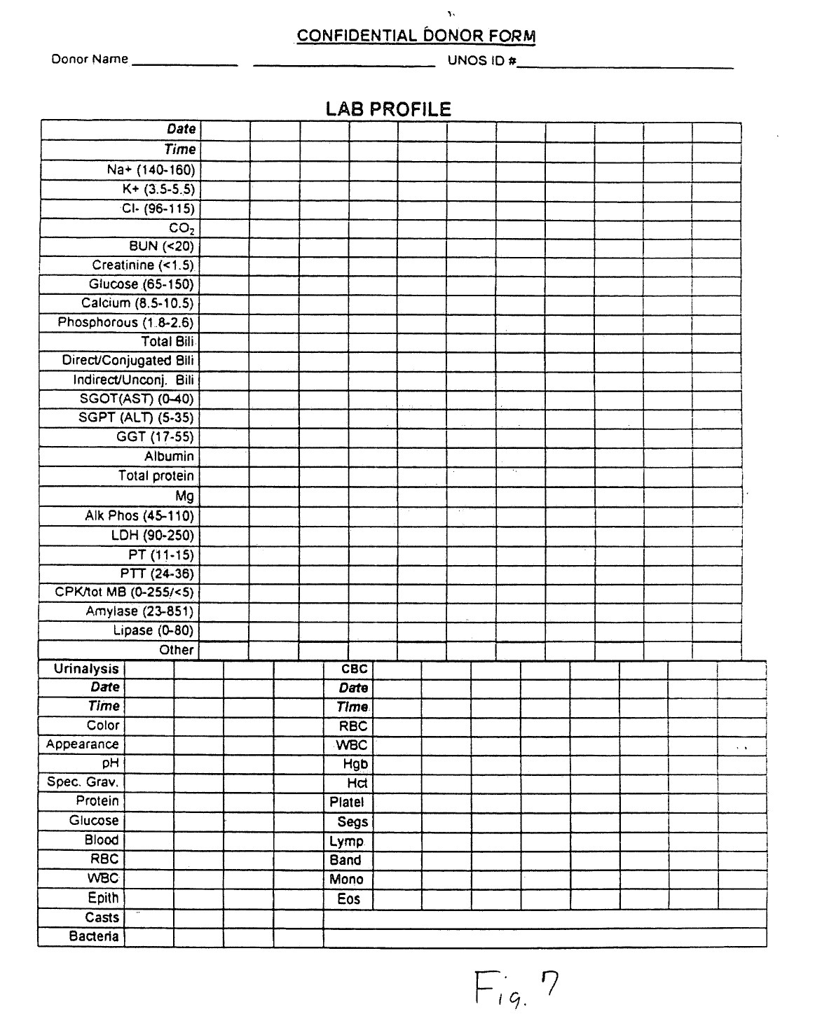

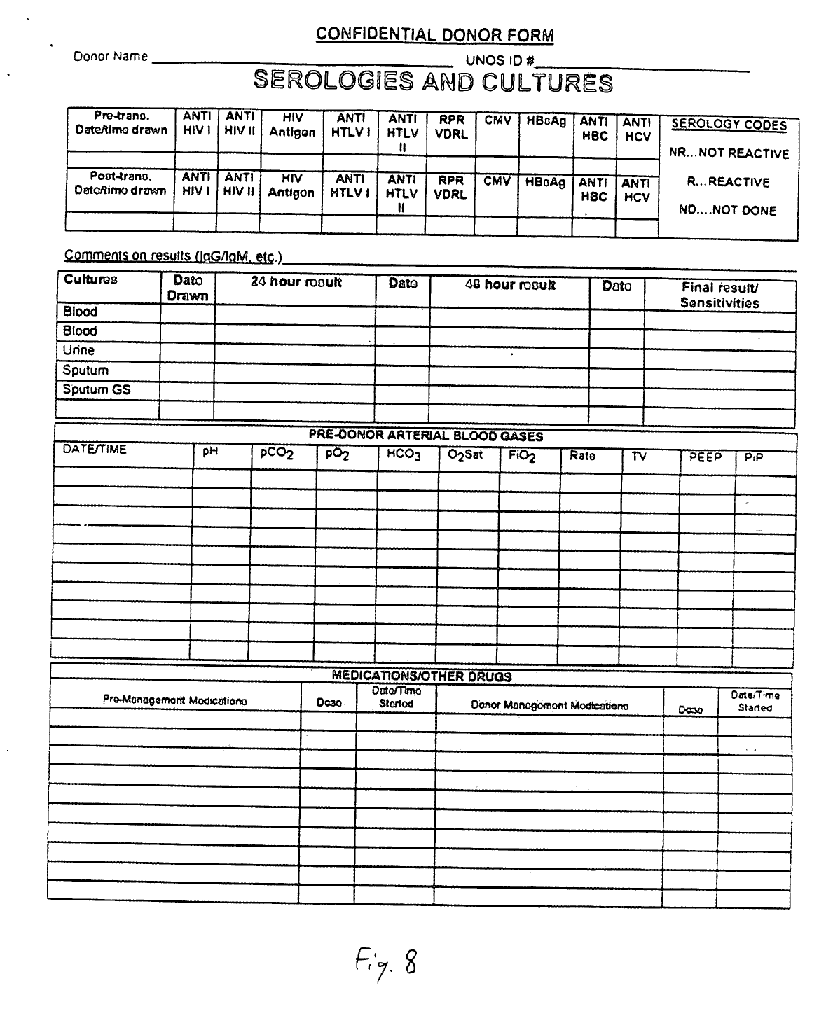

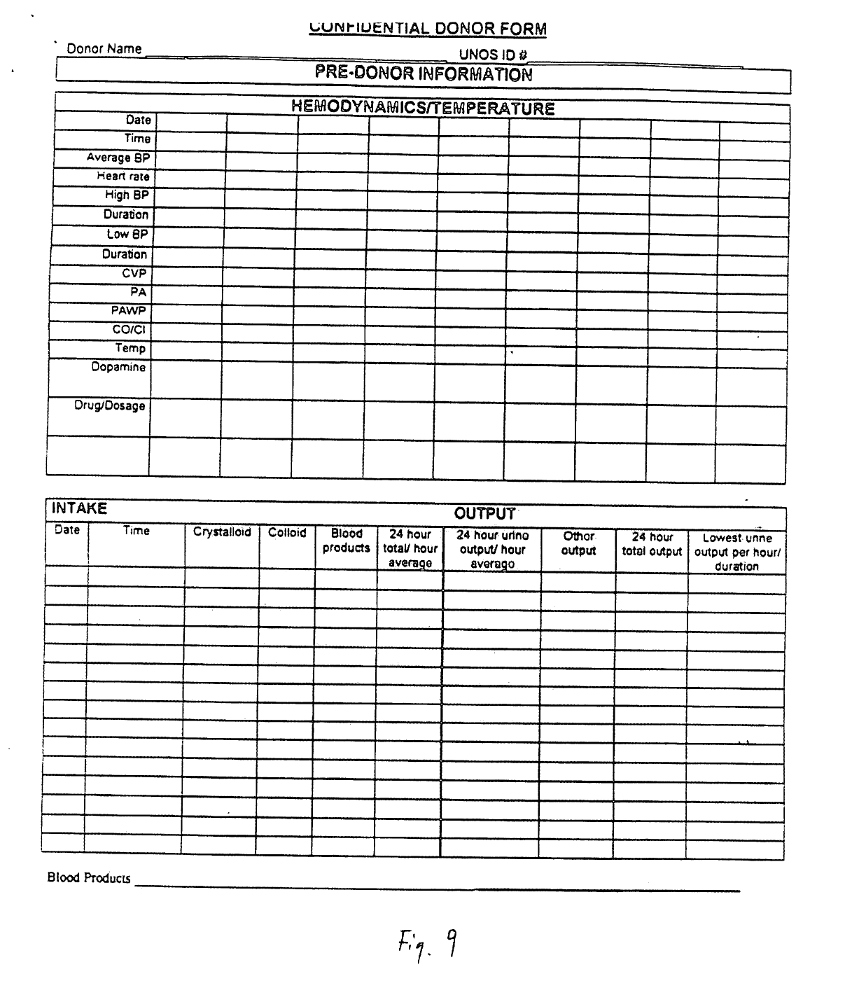

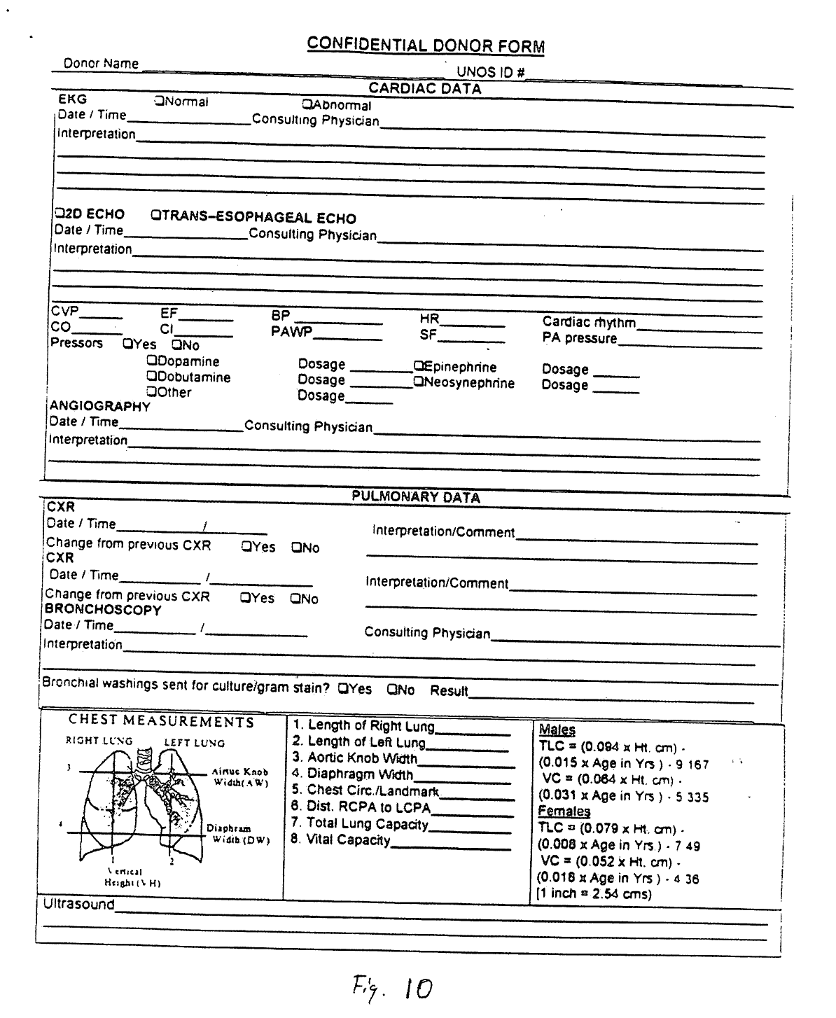

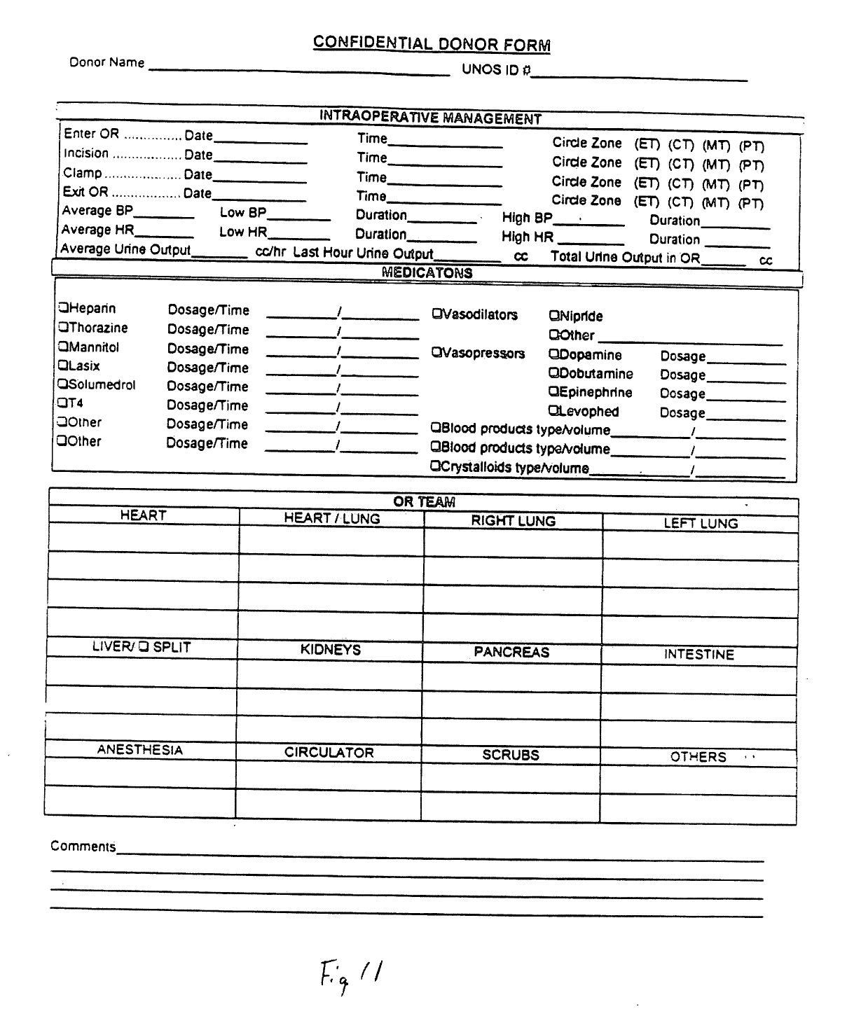

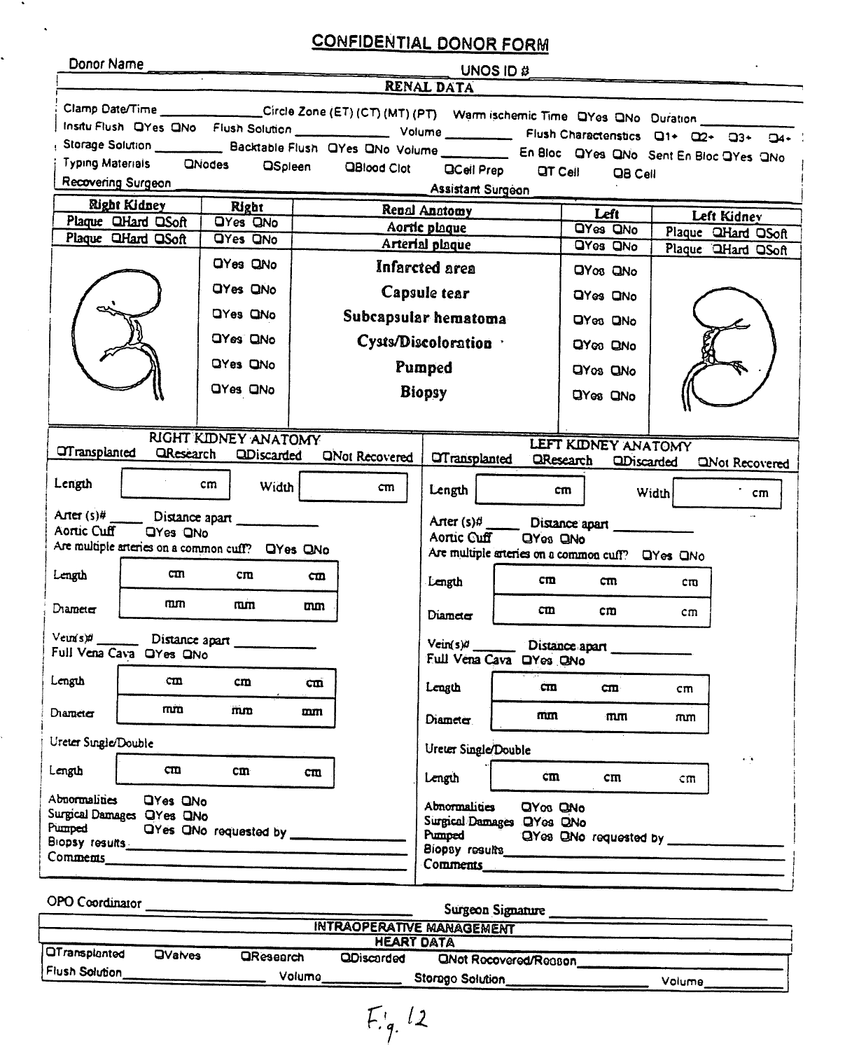

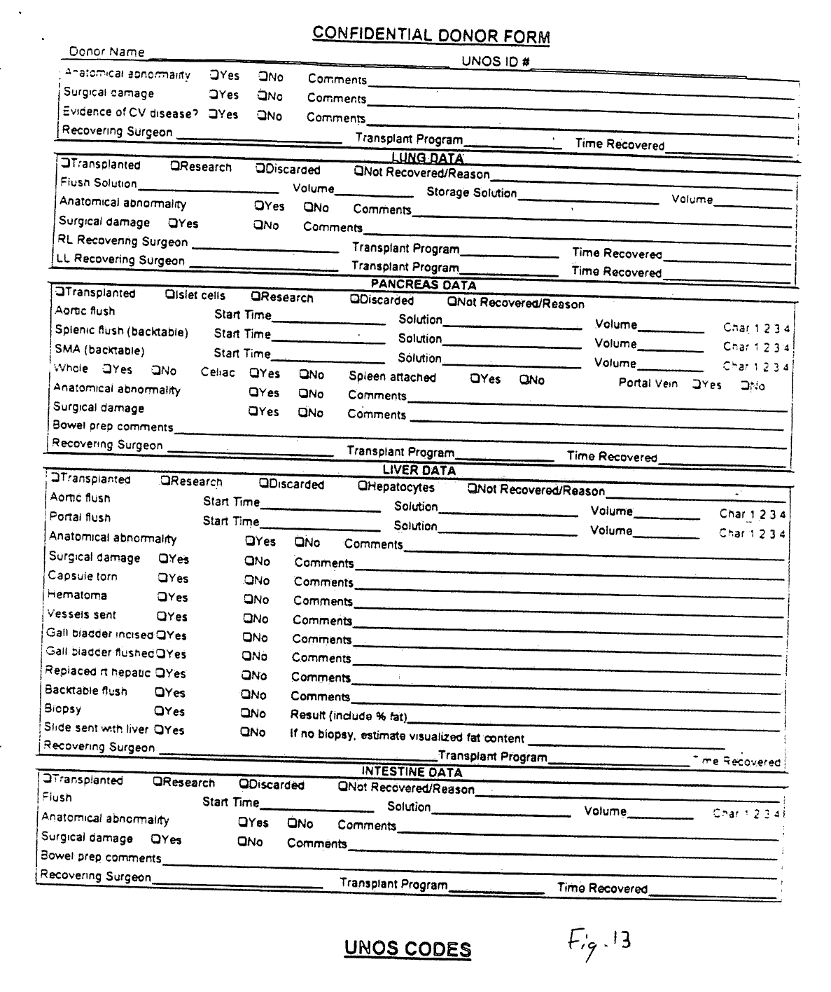

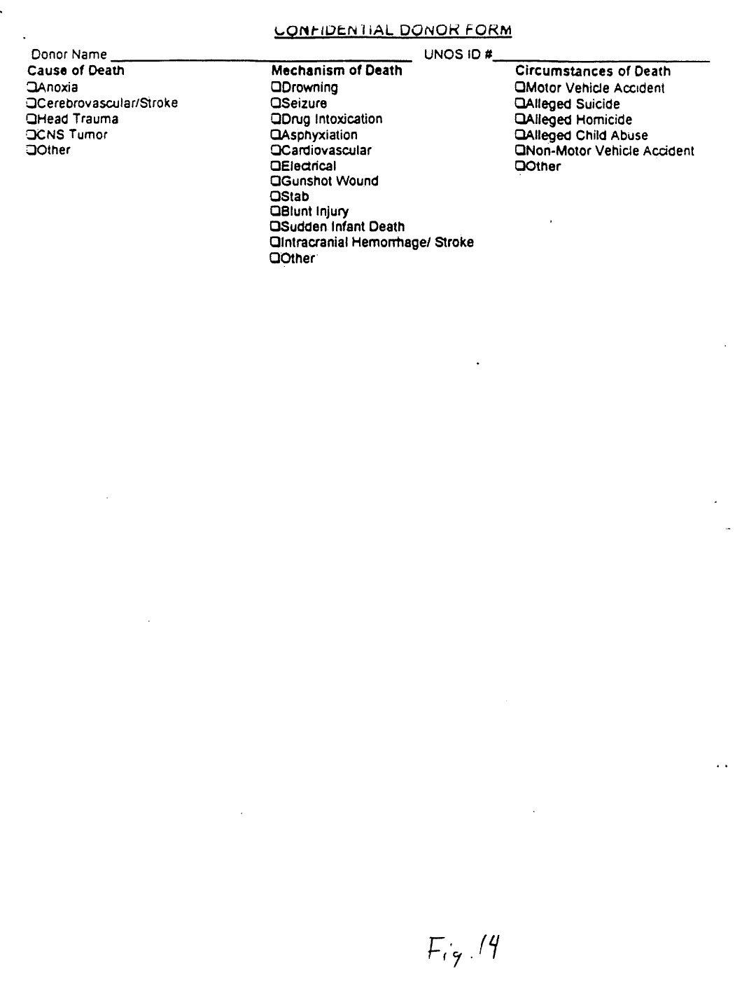

The OPO administrator 130 may also print off active donor records 78; however, inactive donor records may be restricted from being printed. Print selection may be page-based and print out as shown on the electronic donor forms (See FIGS. 4-17). In addition, the OPO administrator application may include an “attachments” window that provides a listing of all attached files (images, video, etc.) for a particular donor record, where the attached files may be shown in icon form. Attachments may be viewed by clicking on an icon if in icon form, or may be otherwise opened using conventional file accessible features, after which the attachment file is closed and the icon or other representation of such file reappears on the attachment listing screen. When an attachment is selected to be viewed, a check of the subscription status of the inquiring organization (generally an OPO) is performed. If the requesting organization does not have a current subscription, access to that attachment may be restricted.

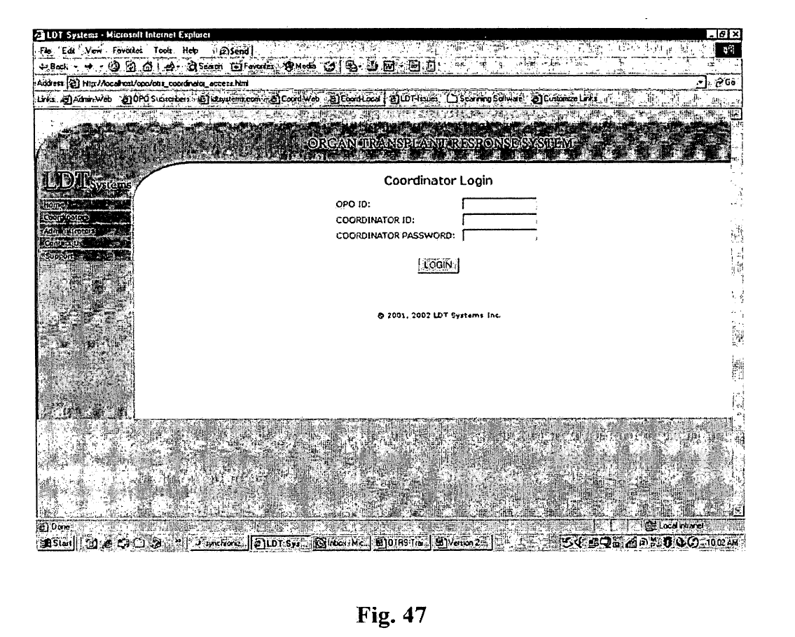

Referring to FIG. 47, a PTC may open the OTRS software application and attempt to sign-on to the OTRS data center 42. By opening the OTRS local application, the software may automatically access the secure network or the LDT Systems Internet site and upload to the OTRS data center 42 the sign-on data for that particular OPO. The OTRS central software will prompt the PTC for a password and user identifier enabling access to donor charts for which that particular PTC is responsible. (Only the OPO admin has full access to all records.) This sign-on data must match the sign-on data embedded within each computer pre-registered to the OPO by OTRS. It is also within the scope of the invention that the sign-on screen may include listings for first name, last name, password, and OPO ID (with the OPO ID defaulting to that OPO to which the laptop is registered).

Referencing FIG. 47-49, the PTC 20 may add a new donor chart or review a list of one or more donor charts associated with that particular PTC that have been previously uploaded to the OTRS data center 42 after signing-on. Each donor chart 78 may have a unique OPTN ID, an Internet password unique to a donor, the donor hospital, the PTC's name, the status of the donor record (active or inactive), and the date last modified associated therewith. Accessing more detailed information is achieved by clicking on the record of choice for viewing and/or changing/updating one or more data entries. Each of the records identified as “active” may be viewed, modified, or printed. “Inactive” records cannot be viewed by non-OPO personnel but may be viewed by the PTC 20 and the OPO administrator 130, with modifications or deletions to be performed by the OPO administrator 130.

The PTC 20 may create a new donor chart by opening the default electronic Organ Donor Forms (ODF) as shown in FIGS. 4-17. By creating a new donor chart 78, the status of the chart will default to “active” status. The OPO ID, OPO contact information (phone, fax, email, etc.), PTC ID, and PTC contact information (phone, fax, email, etc.) will likewise be filled in automatically based upon the information supplied by the PTC 20 during the sign-on procedure to the local OTRS software application, or may simply be automatically filled in based upon embedded information when the local OTRS software application is activated. In either instance, the OPO ID, OPO contact information, PTC ID, and PTC contact information cannot be modified by the PTC 20 after signing on. In addition, the PTC's name and/or initials will be added as constants to the new donor chart.

When adding a donor chart 78, each chart will have a unique random password generated by the local or the central OTRS application to restrict accessibility to only authorized persons. Such restricted access may be limited to the Internet, a secure network gateway, or the local computer 56, 84 upon which the local records 78 are stored. OPO administrators 130 can view the OPTN ID and donor password at all times while they are viewing a particular donor chart 78.

As discussed above, after the donor chart 78 has been uploaded to the OTRS data center 42, the PTC will place a phone call to a particular OPO and read off the OPTN ID and donor chart password that appears on the PTC's local OTRS application screen. Both subscribing and non-subscribing OPO users will need to enter the OPTN ID and donor chart password to access that particular chart via the OTRS data center 42, which may require accessing the Internet. Thereafter, the personnel reviewing the donor charts 78 will make a decision regarding the acceptance of the organ or tissue and in exemplary form follow the above outlined process.

Referencing FIG. 63, in an alternate exemplary embodiment, OTRS 10′, also known as the Tissue Information Processing System (TIPS), may be utilized for tissue donation purposes. For instance, a potential donor may not have one or more organs in a condition sufficient for transplantation, however, the potential donor may still be a viable tissue donor. In addition, a donor may have one or more organs sufficient for transplantation, but wish only to be a tissue donor and not an organ donor. In general, the number of tissue donors is substantially greater than the number of organ donors, at least in part, because the considerations including cause of death and time requirements for tissue donation are not as stringent as for organ donation. An OPO call center or a tissue recovery agency may receive a telephone call from a donor hospital 12′ indicating that a potential tissue donor is available.

Tissue recovery generally, unlike organ recovery, is not critically time sensitive. An exception to such a time lapse might include recovery of tissue segments for use in sports medicine. Prior art techniques devoted a considerable amount of time to qualitative analysis of the donor and tissue consented for donation prior to submission to a tissue processing agency. Such analysis involved the exchange of tangible documentation that, if incorrect, was sent back to the originating party for correction, and then returned to the receiving party for review and verification as to the accuracy of the information contained therein. In accordance with principles of the present invention, OTRS 10′ streamlines the qualitative analysis of the donor and consented tissue by providing all relevant donor data in a donor chart 78′ regarding the consented tissue in a centralized location and in an electronic form, the OTRS data center 42′.

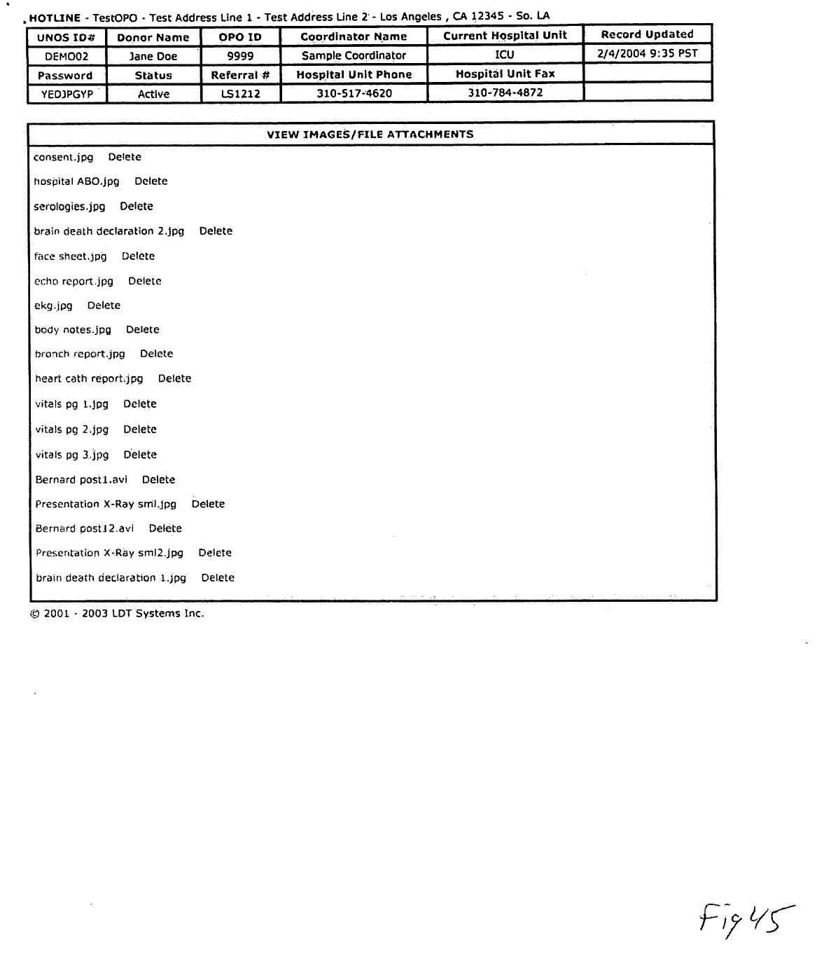

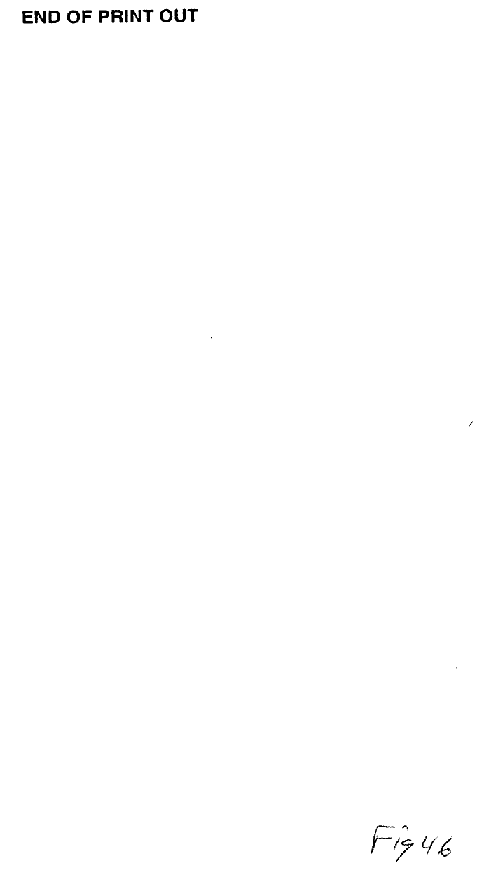

A Tissue Procurement Coordinator (TPC) or Tissue Transplant Coordinator 20′ may be dispatched to the donor hospital 12′ to assess what tissue may be recovered from the potential tissue donor. As discussed above, OTRS 10′ may provide hardware such as a laptop computer 56′ with wireless Internet access and software adapted to interface with the OTRS data center 42′. The hardware and software are adapted to allow the TPC 20′ to enter relevant data into an electronic donor record 78′. As discussed above, such a donor chart or donor record 78′ may include, without limitation, electronic forms (See FIGS. 18-46), consent forms, medical/social questionnaires, diagnostic test results, lab test results, any relevant scanned images and photographs, and any relevant electronic attachments. The donor record 78′ may also include relevant information regarding the tissues donated as shown in exemplary paper form by FIGS. 15-17. If paper forms are utilized, the hardware provided by OTRS 10′ may include an image scanner to convert the paper forms into an electronic form. The information comprising the donor record 78′ may be locally saved on the laptop computer 56′ and/or may be uploaded to or downloaded from the OTRS data center 42′ as discussed above in exemplary form. The OTRS 10′ software would create a unique password and ID for each tissue donor record 78′ and allow an authorized party to access the OTRS data center 42′ and retrieve donor records 78′ for which the authorized party is in possession of the unique IDs and corresponding passwords.

A representative of a Local Tissue Office (LTO) 140 or an OPO may submit an electronic inquiry 142 to the OTRS data center 42′ to access a donor record 78′. It is to be understood that a sign-on protocol would precede any access to a donor record 78′ to verify the authenticity of the party or representative attempting to access the OTRS data center 42′. As discussed above, this may include entering a unique ID and random password for a particular tissue donor to limit LTO representative access to only those records 78′ for which the LTO 140 has the unique IDs and corresponding passwords. The tissue donor record 78′ is put through a quality assurance process that may include a medical director at that LTO 140 approving the tissue donor record 78′. The LTO representative may then review the tissue donor record 78′ again to double check the sufficiency of the record and the suitability of the donor and consented tissue for processing by a Tissue Processing Center 144. In a further exemplary embodiment, the LTO representative may certify the quality of one or more records by providing an electronic certification that attaches to the certified record 78′ that indicates the LTO representative that certified the record, the LTO 140 associated with the LTO representative certifying the record, and the time of the certification.

It is also within the scope of the invention that a donor hospital 12′ representative compile the relevant tissue donor record 78′ in lieu of the TPC 20′, as well as conduct the quality assurance process regarding the donor record 78′. In such an exemplary circumstance, the donor hospital 12′ may be equipped with OTRS 10′ hardware that may include a computer, associated software, and other hardware, such as, without limitation, an image scanner. In this manner, a representative from the donor hospital may enter relevant donor data into the donor record 78′, which may include the transfer of information already within a system of the hospital or other network available to the hospital. In such an exemplary embodiment, the hospital may locally save the donor record 78′ on a local computer and/or database, as well as upload the donor record 78′ to the OTRS data center 42′ as discussed above in exemplary form. Authorized hospital representatives would also have access to download one or more donor records 78′ from the OTRS data center 42′ to perform a quality assurance process analogous to the LTO quality assurance process discussed above. The OTRS 10′ software would create a unique password and ID for each tissue donor record 78′ and allow representatives of the hospital to access the OTRS data center 42′ and retrieve donor records 78′ for which the hospital had the unique IDs and corresponding passwords.

Both LTOs 140 and donor hospitals 12′ are freely able to contract with Tissue Processing Centers 144 for the rights to consented tissue. In an exemplary process, a representative of a Tissue Processing Center 144 may submit an electronic inquiry 146 to the OTRS data center 42′ to access one or more donor records 78′ or certified donor records 78′ for which a contracting LTO 140 or donor hospital 12′ has provided the unique IDs and corresponding passwords. It is to be understood that a sign-on protocol would precede any access to a donor record 78′ to verify the authenticity of the party or Tissue Processing Center representative attempting to access the OTRS data center 42′. As discussed above, this may include a unique ID and password for a particular tissue donor record 78′ to limit Tissue Processing Center representatives from accessing records 78′ for which the unique IDs and corresponding passwords are not available. The records 78′ are reviewed by the Tissue Processing Center representative to discern if the relevant information is present, the quality standards for the tissue are met, and any other requirements are met. If the donor record 78′ is approved by the Tissue Processing Center representative, a request is made by the Tissue Processing Center representative to deliver the approved tissue to the Tissue Processing Center 144. The donated tissue is transported from the donor hospital 12′ to the Tissue Processing Center 144 and the tissue donor record 78′ may be exported to a Tissue Processing Center database 148 that is accessible to one or more Tissue Transplant Hospitals (TTH) 150. The Tissue Processing Center may also maintain records regarding the qualitative control of the acquired tissue as well as an inventory of the acquired tissue that is accessible by Tissue Processing Center representatives and intended end users such as Tissue Transplant Hospital representatives and other medical facility representatives. In a further exemplary embodiment, the Tissue Processing Center representative may certify the quality of one or more records by providing an electronic certification that attaches to the certified record 78′ that indicates the Tissue Processing Center representative that certified the record, the Tissue Processing Center 144 associated with the Tissue Processing Center representative certifying the record, and the time of the certification.

A TTH representative may submit an electronic inquiry 152 to the OTRS data center 42′ and/or an electronic inquiry 154 to the Tissue Processing Center database 148 to access one or more donor records 78′ as well as qualitative and inventory information regarding available tissue. It is to be understood that a sign-on protocol to the OTRS data center 42′ would precede any access to a donor record 78′ to verify the authenticity of the party or representative attempting to access the OTRS data center 42′. As discussed above, this may include a unique ID and random password for a particular donor record to limit TTH representative access to only those records 78′ for which the TTH has the unique IDs and corresponding passwords. The TTH representative may then review one or more records 78′ and discern if the tissue available for transplant meets one or more predetermined criteria for transplant into a potential tissue recipient. The TTH may review the donor record 78′ and request tissue segments appropriate for their patient/recipient. It is also possible that a TTH representative may make a request for one or more tissue segments, where the tissue segments will be selected by a Tissue Processing Center representative that are appropriate for the TTH request.

In accordance with the present invention, the review of tissue data within the OTRS data center 42′ may be done virtually anywhere through the Internet and/or through a private network. Exemplary devices amendable to access the OTRS data center 42′ include, without limitation, cellular telephones, portable digital assistants including Blackberrys, wired or wireless computers having Internet access, or connected to a network other than the Internet through which the OTRS data center 42′ may be accessed.

In a further exemplary embodiment, the tissue donor records 78′ within the OTRS data center 42′ are searchable via one or more search criteria, such as, without limitation, date of recovery, type of tissue recovered, and age of tissue donor.

In an additional exemplary embodiment, OTRS 10′ includes a qualitative analysis of donated organs and/or tissue. The OTRS data center 42′ may include a time log that includes when the organ and/or tissue was accepted by a Tissue Processing Center, when the organ and/or tissue was recovered by a transplant recovery team from the LTO or donor hospital, when the organ and/or tissue was processed by the Tissue Processing Center, what processed tissue segments were put in inventory for use, when the resulting tissue segment(s) were accepted for transplant by a TTH, as well as a recipient log that includes the recipient's name and/or tissue segments received. This log may be updated over time to track the success rate of transplanted organs and/or tissue and the longevity of such organs and/or tissue.

It is to be understood that the above disclosure is exemplary in nature. For instance, it is within the scope of the invention that the exemplary donor forms shown in FIGS. 4-46 may be amended to change the fields of information solicited thereon, as well as the orientation of the fields with respect to one another, as well as the order of the fields with respect to one another. Further, the schematic diagrams of FIGS. 1, 2, 3, and 63 are exemplary in nature and additional steps and apparatuses may be included that have not been discussed herein for purposes of brevity, and likewise, such steps may not necessarily be performed in the exact sequence as discussed herein, and still further, two or more steps shown thereon and/or discussed herein may occur simultaneously.

Following from the above description and invention summaries, it should be apparent to those of ordinary skill in the art that, while the methods and apparatuses described herein constitutes exemplary embodiments of the present invention, the present invention is not limited to these precise embodiments and changes may be made without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any one of the claims, since the invention is defined by the claims and since inherent and/or unforeseen advantages of the present invention may exist even though they may not have been explicitly discussed herein.

1. A method of accessing transplant donor data from a remote location, the method comprising the steps of:
accessing a database over a network containing transplant donor data that includes information specific to a potential transplant donor;
reviewing the information specific to the potential transplant donor; and
acting on the transplant donor data reviewed to establish qualification to at least one of an organ and a tissue available for transplant.

2. The method of claim 1, wherein at least one of the accessing step and the reviewing step includes the step of utilizing a donor specific identification to access and view transplant donor data.

3. The method of claim 2, wherein the donor specific identification is based at least in part upon donor registration identification provided by a centralized organ sharing organization.

4. The method of claim 3, wherein the centralized organ sharing organization maintains a potential transplant recipient list.

5. The method of claim 1, wherein:
the reviewing step includes the step of reviewing information specific to the potential transplant donor that includes at least one of text and images pertaining to at least one of the organ and the tissue available for transplant; and
the network through which the information specific to the potential transplant donor is reviewed includes the Internet.

6. The method of claim 5, wherein the step of reviewing information specific to the potential transplant donor is carried out at least in part by using a microprocessor device.

7. The method of claim 5, wherein the information specific to the potential transplant donor includes at least one of organ availability, approximate dimensions of an organ, blood type of the potential transplant donor, lab results pertaining to an organ, age of the potential transplant donor, and location of the potential transplant donor.

8. The method of claim 1, wherein the reviewing step includes at least one of viewing the transplant donor data at a remote location and downloading the transplant donor data to a remote location.

9. The method of claim 8, wherein the reviewing step includes the step of forwarding at least some of the transplant donor data to transplant personnel having access to a microprocessor device.

10. The method of claim 9, wherein the microprocessor device includes at least one of a personal digital assistant, a wireless telephone, a wired telephone, a wired computer, a wireless computer, a wireless pager, and a wireless book.

11. The method of claim 10, wherein the microprocessor device is amendable to at least one of transmit and receive information to the remote location.

12. The method of claim 11, wherein:
the microprocessor device includes the ability to display images; and
the images include at least one of videos, pictures, X-rays, scanned images, cardiac catheterizations, bronchoscopies, ultrasound images, ultrasound videos and electrocardiograms.

13. The method of claim 1, wherein the acting step includes the step of accepting or declining at least one of the organ and the tissue available for transplant.

14. The method of claim 13, wherein the step of accepting or declining at least one of the organ and the tissue available for transplant is accomplished by at least one of a telephone call to an organ procurement organization representative, a reservation uploaded to the database viewable by an organ procurement organization representative, a telephone call to a donor hospital, a reservation uploaded to the database via the Internet and viewable by an organ procurement organization representative, and a reservation uploaded to a donor hospital database viewable by at least one of an organ procurement organization representative and a donor hospital representative.

15. The method of claim 13, wherein the information specific to a potential transplant donor includes at least one of organ availability, approximate dimensions of an organ, blood type of the potential transplant donor, lab results pertaining to an organ, age of the potential transplant donor, and location of the potential transplant donor.

16. The method of claim 1, wherein:
the accessing step includes the step of accessing an Internet site containing transplant donor data;
the accessing step includes providing a unique access identifier prior to the reviewing step; and
the reviewing step includes at least one of viewing the transplant donor data at a remote location and downloading the transplant donor data to a remote location.

17. The method of claim 16, wherein:
the unique access identifier includes at least one of identification of a person accessing the transplant donor data and identification of a specific transplant donor data record to be reviewed; and
the reviewing step includes reviewing the transplant donor data by transplant center personnel.

18. The method of claim 1, further comprising the step of receiving authorization code data for logging onto the network to access and review transplant donor data, where the authorization code is specific to the potential transplant donor.

19. The method of claim 18, wherein the accessing step includes the step of utilizing the authorization code to access the network and review the transplant donor data specific to the potential transplant donor.

20. The method of claim 1, further comprising the step of receiving an electronic offer notification pertaining to at least one of the organ and the tissue utilizing at least one of a personal digital assistant, a wireless telephone, a wired telephone, a wired computer, a wireless computer, a wireless pager, and a wireless book.

21. The method of claim 20, wherein the electronic offer notification includes at least one of an Internet address associated with the database and transplant donor data.

22. The method of claim 21, wherein the electronic offer notification includes an Internet address associated with the database and an authorization code to access the transplant donor data.

23. A method of gathering and inputting transplant donor data to a database, the method comprising the steps of:
compiling a transplant donor record specific to a transplant donor;
accessing a remote database capable of storing a plurality of transplant donor records; and
uploading the transplant donor record to the remote database.

24. The method of claim 23, wherein the compiling step includes the step of inputting transplant donor data into at least one of a tangible expression and a digital expression.

25. The method of claim 24, wherein the transplant donor data is input into a series of relevant fields that include at least one of transplant donor blood type, time of death of the transplant donor, cause of death of the transplant donor, transplant donor lab results, the transplant donor's organs available for transplant, age of the transplant donor, and dimensions of the transplant donor's organs available for transplant.

26. The method of claim 24, wherein the compiling step includes the step of inputting transplant donor history data that includes at least one of transplant donor illnesses or disorders, transplant donor medical treatments, transplant donor allergies, transplant donor exposure to toxic substances, transplant donor smoking habits, transplant donor drinking habits, transplant donor medications, transplant donor risky sexual behavior, transplant donor drug usage, and transplant donor blood products received.

27. The method of claim 24, wherein the compiling step includes transforming the tangible expression of the transplant donor data into the digital expression of the transplant donor data.

28. The method of claim 25, wherein the step of inputting transplant donor data into a series of fields includes inputting the digital expression of the transplant donor data utilizing at least one of a wireless data input device and a wired data input device.

29. The method of claim 28, wherein:
the step of inputting the transplant donor data into a series of fields includes the step of providing an electronic donor data form;
the series of fields include at least one of transplant donor name, transplant donor address, transplant donor next of kin name, and transplant donor next of kin address; and
the electronic donor forms reside in memory on at least one of the wireless data input device and the wired data input device.

30. The method of claim 23, wherein the compiling step includes providing a computer that may access an electronic form adapted to include transplant donor data.

31. The method of claim 23, wherein:
the step of uploading includes uploading pure data in a first set of predetermined fields comprising the transplant donor record; and
the pure data of the transplant donor record is adapted to be extracted from the first set of predetermined fields and assimilated with a second set of predetermined fields associated with the remote database.

32. The method of claim 23, wherein:
the accessing step includes the step of viewing at least one uploaded transplant donor record; and
the viewing step includes viewing the transplant donor data from the remote database.

33. The method of claim 23, wherein the accessing step includes connecting to the remote database utilizing the Internet.

34. The method of claim 23, further comprising the step of signing onto the remote database.

35. The method of claim 34, wherein the step of signing onto the remote database includes the step of activating software installed on a digital device to connect to the remote database.

36. The method of claim 35, wherein the digital device includes at least one of a wireless personal digital assistant, a wireless computer, a wired computer, a wireless telephone and a wired telephone.

37. The method of claim 35, wherein the step of activating software installed on the digital device automatically attempts to connect to the remote database and download from the remote database pre-registered sign-on data particular to at least one of a procurement organization representative and the digital device.

38. The method of claim 37, wherein the digital device is adapted to download at least one of transplant donor management software additions, transplant donor management software updates, and transplant donor management software deletions to the remote computer via wireless or wireline network connection.

39. The method of claim 37, wherein the digital device is adapted to load at least one of transplant donor management software additions, transplant donor management software updates, and transplant donor management software deletions to the remote computer via wireless or wireline network connection.

40. The method of claim 37, wherein the pre-registered sign-on data particular to at least one of the procurement organization representative and the digital device must match unique sign-on data specific to at least one of the procurement organization representative and the digital device.

41. The method of claim 40, wherein:
the remote database is accessible via a secure network;
the pre-registered sign-on data is assigned by a network administrator;
the pre-registered sign-on data includes embedded data within the digital device;
the digital device is pre-registered by the network administrator; and
the procurement organization representative must input a unique identifier prior to accessing the remote database having the plurality of transplant donor records specific to transplant donors.

42. The method of claim 41, wherein the unique identifier includes at least one of a first name, a last name, a password, and a procurement organization representative identifier.

43. The method of claim 23, wherein the compiling step includes the step of inputting transplant donor data in to a first set of predetermined fields comprising the transplant donor record.

44. The method of claim 43, wherein the uploading step includes the step of facsimile transmitting the transplant donor record.

45. A method of gathering and displaying transplant donor data, the method comprising the steps of:
compiling a transplant donor record specific to a transplant donor;
transmitting an electronic version of the transplant donor record; and
displaying the transplant donor record using the electronic version of the transplant donor record.

46. The method of claim 45, wherein the transmitting step includes at least one of facsimile transmitting the transplant donor record via telephone communication and electronic data transmitting the transplant donor record via computer network communication.

47. The method of claim 45, wherein the compiling step include compiling the electronic version of the transplant donor record.

48. The method of claim 45, wherein the displaying step includes displaying the transplant donor record on a tangible medium.

49. A method of organizing and making available transplant donor data, the method comprising the steps of:
providing a secure database to store transplant donor data;
providing access to a selective third party to at least one of upload and view transplant donor data and download and view transplant donor data; and
generating an authorization code required to access the transplant donor data by the selective third party.

50. The method of claim 49, wherein the step of providing a secure database includes the step of providing a secure server operatively coupled to a secure network.

51. The method of claim 50, wherein the secure network is at least one of a private network and a public network.

52. The method of claim 49, wherein the step of providing access to the selective third party includes the step of providing access to a procurement organization representative.

53. The method of claim 49, wherein the step of providing access to the procurement organization representative includes the step of providing customizing options to a network administrator of a procurement organization.

54. The method of claim 53, wherein the customizing options include at least one of the ability to add or delete procurement organization representatives from a selective third party list, the ability to amend medical history questions pertaining to a transplant donor, the ability to amend social history questions pertaining to a transplant donor, the ability to amend information regarding a transplant donor hospital, and the ability to view updated listings for a transplant donor hospital

55. The method of claim 53, wherein the customizing options include at least one of the ability to amend a name of an individual receiving a notification that a new transplant donor has been added to the secure database, the ability to amend contact information of an individual receiving a notification that a new transplant donor has been added to the secure database, and the ability to amend how an individual receives a notification that a new transplant donor has been added to the secure database.

56. The method of claim 49, wherein the authorization code is randomly generated by software accessing the secure database.

57. The method of claim 56, wherein:
the step of providing access to the selective third party to upload and view transplant donor data includes the step of providing a transplant center representative with access to the secure database to review transplant donor data upon authentication; and
the authentication includes the transplant center representative providing the authorization code to gain access to the secure database.

58. The method of claim 49, wherein the secure database includes at least one of text and images.

59. The method of claim 58, wherein the text of the transplant donor data includes at least one of consent information, transplant donor name, transplant donor organs recovered and transplant donor tissue recovered.

60. The method of claim 58, wherein the images of the transplant donor data include at least one of videos, pictures, X-rays, scanned images, cardiac catheterizations, bronchoscopies, ultrasound images, ultrasound videos and electrocardiograms.

61. The method of claim 49, further comprising the step of tracking access by the selective third party to discern when transplant donor data has been at least one of viewed, updated, uploaded, and downloaded, wherein the tracking access step includes the step of providing a network administrator to oversee the secure database.

62. The method of claim 61, wherein the network administrator may restrict access to the selective third party to less than all of the transplant donor data stored on the secure database.

63. The method of claim 49, wherein the generating step includes the step of providing at least one of hardware and software to generate the authorization code on a transplant donor specific basis.

64. The method of claim 63, further comprising the step of registering at least one of the hardware and the software, wherein the step of registering includes a network administrator issuing at least one of the hardware and the software, wherein at least one of the hardware and the software include a unique identifier.

65. The method of claim 64, further comprising the step of tracking access by the selective third party to discern when transplant donor data has been at least one of viewed, updated, uploaded, and downloaded, wherein the tracking access step includes the step of tracking the unique identifier by the network administrator.

66. The method of claim 49, further comprising the step of sending an electronic offer notification to at least one of a personal digital assistant, a wireless telephone, a wired telephone, a wired computer, a wireless computer, a wireless pager, and a wireless book associated with the selective third party.

67. The method of claim 66, wherein the electronic offer notification includes at least one of an Internet address associated with the secure database and transplant donor data.

68. The method of claim 49, further comprising the step of providing electronic forms into which the transplant donor data may be entered.

69. The method of claim 68, wherein:
the providing electronic forms step includes the step of synchronizing the electronic forms with the secure database; and
the providing access step includes the step of uploading at least one of the electronic forms and the transplant donor data to the secure database.

70. The method of claim 49, wherein the selective third party includes at least one of an organ procurement organization representative and a transplant center representative.

71. The method of claim 70, wherein at least one of the organ procurement organization representative and a transplant center representative accesses the secure database to at least one of import data thereto and export data therefrom.

72. A method of gathering and inputting transplant donor data to a database in the form of a pure data system, the method comprising the steps of:
providing a series of data input options into which transplant donor data may be input to create an transplant donor record, the series of data input options including at least one of edit transplant donor data, update transplant donor data, delete transplant donor data, and submit transplant donor data;
inputting transplant donor data into at least one of the series of data input options to create the transplant donor record;
transmitting the transplant donor record to an transplant donor database; and
availing the transplant donor record via the Internet.

73. The method of claim 72, wherein the inputting step includes providing a wireless storage device capable of storing the transplant donor data within the data input options to create the transplant donor record.

74. The method of claim 73, wherein the wireless storage device includes at least one of a wireless computer, a wireless personal digital assistant, and a wireless phone.

75. A method of gathering transplant donor data, the method comprising the steps of:
utilizing a computer operatively coupled to a scanner, where the computer has at least one electronic transplant donor form adapted to be manipulatable to input transplant donor data using at least one of keystrokes, digital handwriting, and scanned images; and
the computer includes software to facilitate the uploading of the transplant donor data to a remote database over a network connection, the remote database including a remote digital processing device, such that the remote database is accessible by an intended third party.

76. A transplant information system to facilitate the dissemination of information pertaining to transplantable organs and tissue from a donor hospital to a transplant center, the system comprising:
a database accessible by a procurement organization representative and a transplant center representative, the database including transplant donor data; and
a secure network through which the transplant donor data is accessed by at least one of the procurement organization representative and the transplant center representative.

77. The transplant information system of claim 76, wherein the procurement organization representative is equipped with wireless hardware that includes at least one of a cellular telephone, a personal digital assistant, a laptop computer, and a handheld computer.

78. The transplant information system of claim 77, wherein:
the wireless hardware includes software adapted to compile a transplant donor data record via data manipulation; and
the transplant donor record includes at least one of text and images.

79. The transplant information system of claim 78, wherein the transplant donor data record includes electronic forms divided into predetermined data fields adapted to receive transplant donor data entered by the procurement organization representative.

80. The transplant information system of claim 79, wherein the wireless hardware includes software adapted access the database via the secured network.
